(19) United States
(12) Patent Application Publication
(10) Pub. No.: US 2025/0352534 A1
(43) Pub. Date: Nov. 20, 2025

(54) Use of Niraparib for the Treatment of Brain Cancer

(71) Applicant: Tesaro, Inc., Wilmington, DE (US)

(72) Inventors: Nader Sanai, Phoenix, AZ (US); Shwetal Mehta, Phoenix, AZ (US)

(21) Appl. No.: 18/838,460

(22) PCT Filed: Feb. 15, 2023
(86) PCT No.: PCT/US2023/062661

§ 371 Date: Aug. 14, 2024

Related U.S. Application Data
(60) Provisional application No. 63/268,051, filed on Feb. 15, 2022.
(60) Provisional application No. 63/426,589, filed on Nov. 18, 2022.

(51) Int. Cl. A61K 31/454; A61K 31/495; A61K 31/502; A61N 5/10; A61P 35/00; A61P 35/04

(52) U.S. Cl. CPC A61K 31/454; A61K 31/495; A61K 31/502; A61N 5/10; A61P 35/00; A61P 35/04

(57) ABSTRACT

The present invention provides methods of administering niraparib for the treatment of primary and metastatic brain cancer.

This application is a National Stage Application under 35 U.S.C. § 371 and claims the benefit of International Application No. PCT/US2023/062661, filed Feb. 15, 2023, which claims priority to U.S. Provisional Application No. 63/268,051, filed Feb. 15, 2022, and U.S. Provisional Application No. 63/426,589, filed Nov. 18, 2022, the disclosures of which are each incorporated herein by reference.

### FIELD OF THE INVENTION

The present invention provides methods of administering niraparib for treatment of primary and metastatic brain cancer.

### BACKGROUND OF THE INVENTION

Cancer is a serious public health problem. Worldwide, an estimated 308,102 people were diagnosed with a primary brain or spinal cord tumor in 2020. The 5-year survival rate for people in the United States with a cancerous brain or CNS tumor is almost 36%. (Cancer Facts & Figures 2022, the ACS website) with 609,640 people in the United States of America dying of cancer in 2018 alone. American Cancer Society, Cancer Facts & Figures 2018 (available at American Cancer Society website). Accordingly, there continues to be a need for effective therapies to treat cancer patients including primary and metastatic brain cancer patients.

### SUMMARY OF THE INVENTION

The present disclosure is directed to a method of treating primary or metastatic brain cancer in a human subject in need thereof, the method comprising administering to the human subject an effective dose of niraparib, or a pharmaceutically acceptable salt thereof.

The present disclosure further provides niraparib, or a pharmaceutically acceptable salt thereof, for use in treatment of primary or metastatic brain cancer in a human subject in need thereof.

The present disclosure further provides niraparib, or a pharmaceutically acceptable salt thereof, in the manufacture of a medicament for treatment of primary or metastatic brain cancer in a human subject in need thereof.

### BRIEF DESCRIPTION OF THE DRAWINGS

FIG. 1 shows a summary of the Phase 0 clinical protocol for niraparib in newly diagnosed glioblastoma (HGG; high grade glioma) and recurrent IDH1/2+ATRX (WHO II-IV glioma).

FIG. 2 shows a summary of the protocol to quantify the effect of P-gp and BCRP in limiting CNS penetration of niraparib.

FIG. 3 shows a summary of the results of quantifying the effect of P-gp and BCRP in limiting CNS penetration of niraparib.

FIG. 4 shows a summary of the Arm A clinical protocol for niraparib in newly diagnosed glioblastoma (HGG; high grade glioma).

FIG. 5 shows a summary of the patient demographics and patient safety profile for Arm A Phase 0 and Expansion studies for niraparib in newly diagnosed glioblastoma (HGG; high grade glioma).

FIG. 6 shows a summary of unbound niraparib levels in non-enhancing tumor tissue in Arm A cohorts 1 and 2.

FIG. 7 shows a summary of unbound niraparib levels in non-enhancing tumor tissue in Arm A cohorts 1 and 2 with mean values for 300 mg dosing and 200 mg dosing for cohort 1, and for 300 mg dosing for cohort 2.

FIG. 8 shows total and unbound tumor/plasma ratios of niraparib Arm A cohorts 1 and 2 in non-enhancing tumor tissue and enhancing tumor tissue.

FIG. 9 shows functional pharmacodynamic data for niraparib including PAR levels after ex vivo radiation compared to untreated samples.

FIG. 10 shows a summary of clinical outcomes in newly diagnosed glioblastoma where Median PFS is depicted in months.

### DETAILED DESCRIPTION

The present disclosure is directed to, inter alia, methods of treating brain cancer in a human subject in need thereof, the method comprising administering to the human subject an effective dose of niraparib, or a pharmaceutically acceptable salt thereof.

The present disclosure is further directed to, inter alia, methods of treating primary or metastatic brain cancer in a human subject in need thereof, the method comprising administering to the human subject an effective dose of niraparib, or a pharmaceutically acceptable salt thereof.

The present disclosure is further directed to methods of treating central nervous system (CNS) cancers in a human subject in need thereof, the method comprising administering to the human subject an effective dose of niraparib, or a pharmaceutically acceptable salt thereof.

In some embodiments, the method of treating brain cancer in a human subject in need thereof comprises:
(i) administering to the human subject an effective dose of niraparib, or a pharmaceutically acceptable salt thereof,
(ii) resection of the brain cancer/tumor;
(iii) determination of concentration of niraparib in the brain cancer/tumor;
(iv) dosing of niraparib, or a pharmaceutically acceptable salt thereof, if sufficient niraparib is determined to be present at step (iii); and
(v) optionally further dosing with niraparib, or a pharmaceutically acceptable salt thereof, as a maintenance treatment.

In some embodiments, the human subject is treated with niraparib, or a pharmaceutically acceptable salt thereof, for 4 days in step (i).

In some embodiments, sufficient niraparib present in step (iii) is an unbound concentration of niraparib >5-fold of the biochemical IC50 value of niraparib. In some embodiments, 5-fold of the biochemical IC50 value of niraparib is 19 nM. In some embodiments, 5-fold of the biochemical IC50 value of niraparib is about 19 nM.

In some embodiments, the human subject is also treated with radiation therapy in step (iv), particularly stereotactic radiation therapy. In some embodiments, the niraparib, or a pharmaceutically acceptable salt thereof, and radiation therapy is administered for about 6-7 weeks. In some embodiments, the niraparib, or a pharmaceutically acceptable salt thereof, is administered as an add-on therapy to radiation therapy in the treatment of brain cancer. In some embodiments, the niraparib, or a pharmaceutically acceptable salt thereof, is administered as an add-on therapy to radiation therapy in the treatment of unmethylated MGMT glioma. In some embodiments, the niraparib, or a pharmaceutically acceptable salt thereof, is administered as an add-on therapy to radiation therapy in the treatment of unmethylated MGMT glioblastoma. In some embodiments, the niraparib, or a pharmaceutically acceptable salt thereof, is administered as an add-on therapy to radiation therapy in the treatment of unmethylated glioma. In some embodiments, the niraparib, or a pharmaceutically acceptable salt thereof, is administered as an add-on therapy to radiation therapy in the treatment of unmethylated glioblastoma.

In some embodiments, the human subject further receives maintenance treatment of niraparib, or a pharmaceutically acceptable salt thereof, without radiation therapy in step (v) after the treatment with niraparib, or a pharmaceutically acceptable salt thereof, and radiation therapy in step (iv).

In some embodiments, the human subject is also treated with radiotherapy, particularly stereotactic radiation therapy. In some embodiments, radiotherapy can include, but is not limited to one or more of the following:
External beam radiation therapy delivers radiation from a machine and through the body to reach metastatic tumors.
   Whole-brain radiation targets the entire brain to hit multiple tumors or any metastatic disease that hides from an MRI scan.
   Stereotactic radiosurgery (e.g., Cyberknife) directs a high dose of radiation targeted to the specific shape of the tumor, sparing surrounding healthy tissue from unnecessary radiation exposure.
   Proton therapy uses protons (instead of X-rays) to treat metastatic brain tumors. Like stereotactic radiosurgery, proton therapy minimizes harm to healthy tissue surrounding a tumor.
Brachytherapy is radioactive material implanted within a tumor to prevent further growth.

In some embodiments, niraparib, or a pharmaceutically acceptable salt thereof, is administered as an add-on therapy to radiation therapy in the treatment of brain cancer. In some embodiments, niraparib, or a pharmaceutically acceptable salt thereof, is administered as an add-on therapy to radiation therapy in the treatment of unmethylated MGMT glioma. In some embodiments, niraparib, or a pharmaceutically acceptable salt thereof, is administered as an add-on therapy to radiation therapy in the treatment of unmethylated MGMT glioblastoma. In some embodiments, niraparib, or a pharmaceutically acceptable salt thereof, is administered as an add-on therapy to radiation therapy in the treatment of unmethylated glioma. In some embodiments, niraparib, or a pharmaceutically acceptable salt thereof, is administered as an add-on therapy to radiation therapy in the treatment of unmethylated glioblastoma.

In some embodiments, the brain cancer is primary or metastatic brain cancer. In some embodiments, the primary or metastatic brain cancer is newly diagnosed. In some embodiments, the primary brain cancer is newly diagnosed. In some embodiments, the metastatic brain cancer is newly diagnosed.

In some embodiments, the human subject is treated with presurgical niraparib, or a pharmaceutically acceptable salt thereof. In some embodiments, the human subject is treated with presurgical niraparib, or a pharmaceutically acceptable salt thereof, prior to surgical resection. In some embodiments, the human subject is treated with presurgical niraparib, or a pharmaceutically acceptable salt thereof, prior to surgical resection, and the concentration of niraparib in the brain cancer tumor is measured post-resection. In some embodiments, the human subject is treated with presurgical niraparib, or a pharmaceutically acceptable salt thereof, for 4 days prior to surgical resection. In some embodiments, the final presurgical dose is administered 3-5 hours or 8-10 hours before tumor resection.

In some embodiments, the brain cancer is primary brain cancer.

In some embodiments, the brain cancer is glioma.

In some embodiments, the brain cancer is high-grade glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

In some embodiments, the administration of niraparib, or a pharmaceutically acceptable salt thereof, and radiation therapy begins after resection of a primary brain cancer tumor. In some embodiments, the niraparib, or a pharmaceutically acceptable salt thereof, is administered as an add-on therapy to radiation therapy in the treatment of brain cancer. In some embodiments, the niraparib, or a pharmaceutically acceptable salt thereof, is administered as an add-on therapy to radiation therapy in the treatment of unmethylated MGMT glioma. In some embodiments, the niraparib, or a pharmaceutically acceptable salt thereof, is administered as an add-on therapy to radiation therapy in the treatment of unmethylated MGMT glioblastoma. In some embodiments, the niraparib, or a pharmaceutically acceptable salt thereof, is administered as an add-on therapy to radiation therapy in the treatment of unmethylated glioma. In some embodiments, the niraparib, or a pharmaceutically acceptable salt thereof, is administered as an add-on therapy to radiation therapy in the treatment of unmethylated glioblastoma.

In some embodiments, the radiation therapy is about 60 Gy (unit gray). In some embodiments, the radiation therapy is about 10 Gy (unit gray).

In some embodiments, the niraparib, or a pharmaceutically acceptable salt thereof, and radiation therapy is administered for about 6-7 weeks.

In some embodiments, the human subject further receives maintenance treatment of niraparib, or a pharmaceutically acceptable salt thereof, without radiation therapy after the treatment with niraparib, or a pharmaceutically acceptable salt thereof, and radiation therapy.

In some embodiments, the human subject further receives maintenance treatment of niraparib, or a pharmaceutically acceptable salt thereof, without radiation therapy subsequent to the treatment with niraparib, or a pharmaceutically acceptable salt thereof, and radiation therapy.

In some embodiments, the human subject further receives maintenance treatment of niraparib, or a pharmaceutically acceptable salt thereof, without radiation therapy subsequent to the treatment with niraparib, or a pharmaceutically acceptable salt thereof, and radiation therapy, until disease progression occurs.

In some embodiments, the human subject further receives maintenance treatment of niraparib, or a pharmaceutically acceptable salt thereof, without radiation therapy about 4 weeks after the treatment with niraparib, or a pharmaceutically acceptable salt thereof, and radiation therapy.

In some embodiments, the administering of niraparib, or a pharmaceutically acceptable salt thereof, follows prior therapy. In some embodiments, the administering of niraparib, or a pharmaceutically acceptable salt thereof, does not follow prior therapy. In some embodiments, the niraparib, or a pharmaceutically acceptable salt thereof, is administered as an add-on therapy to radiation therapy.

In some embodiments, the niraparib, or a pharmaceutically acceptable salt thereof, is administered in a dose that is equivalent to 200 mg or 300 mg of niraparib free base. In some embodiments, the niraparib, or a pharmaceutically acceptable salt thereof, is administered in a dose that is equivalent to about 300 mg of niraparib free base. In some embodiments, the niraparib, or a pharmaceutically acceptable salt thereof, is administered in a single daily dose. In some embodiments, the niraparib, or a pharmaceutically acceptable salt thereof, is administered twice per day. In some embodiments, the niraparib, or a pharmaceutically acceptable salt thereof, is administered in a daily dose that is equivalent to 200 mg or 300 mg of niraparib free base. In some embodiments, the niraparib, or a pharmaceutically acceptable salt thereof, is administered in a daily dose that is equivalent to about 300 mg of niraparib free base. In some embodiments, the niraparib, or a pharmaceutically acceptable salt thereof, is administered in a daily dose that is equivalent to about 200 mg of niraparib free base.

In some embodiments, the niraparib, or a pharmaceutically acceptable salt thereof, is dosed as niraparib tosylate monohydrate.

In some embodiments, the niraparib, or a pharmaceutically acceptable salt thereof, is administered in the form of a tablet.

In some embodiments, the dose of niraparib, or a pharmaceutically acceptable salt thereof, is administered daily.

In some embodiments, the primary brain cancer is selected from the group consisting of anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumour, pinealoma, diffuse intrinsic pontine glioma, IDH1/2(+) ATRX mutant glioma, malignant glioma and primitive neuroectodermal tumor of the brain.

In some embodiments, the primary brain cancer is a WHO grade IV tumor.

In some embodiments, the primary brain cancer is glioblastoma. In some embodiments, the metastatic brain cancer is glioblastoma. In some embodiments, the central nervous system cancer is glioblastoma.

In some embodiments, the glioblastoma is recurrent glioblastoma. In some embodiments, the glioblastoma is a newly diagnosed glioblastoma. In some embodiments, the newly diagnosed or recurrent glioblastoma is associated with IDH-mutation and ATRX loss. In some embodiments, the glioblastoma is primary glioblastoma.

In some embodiments, the human subject has newly diagnosed glioblastoma and MGMT promoter hypermethylation. In some embodiments, the human subject has unmethylated MGMT tumors. In some embodiments, the human subject has unmethylated MGMT promoter. In some embodiments, the human subject has unmethylated MGMT glioma. In some embodiments, the human subject has unmethylated MGMT glioblastoma. In some embodiments, the human subject has unmethylated glioma. In some embodiments, the human subject has unmethylated glioblastoma.

In some embodiments, the primary brain cancer is a glioma. In some embodiments, the metastatic brain cancer is a glioma. In some embodiments, the central nervous system cancer is a glioma. In some embodiments, the glioma is recurrent glioma.

In some embodiments, the glioma is an adult-type diffuse glioma. In some embodiments, the adult-type diffuse glioma is astrocytoma (IDH-mutant), oligodendroglioma (IDH-mutant and 1p/19q co-deleted), or glioblastoma (IDH-wild-type).

In some embodiments, the glioma is a pediatric-type diffuse low-grade glioma. In some embodiments, the pediatric-type diffuse low-grade glioma is selected from diffuse astrocytoma (MYB or MYBL1 altered), angiocentric glioma, polymorphous low-grade neuroepithelia tumor of the young, or diffuse low-grade glioma (MAPK pathway altered).

In some embodiments, the glioma is a pediatric-type diffuse high-grade glioma. In some embodiments, the pediatric-type diffuse high-grade glioma is selected from diffuse midline glioma (H3 K27 altered), diffuse hemispheric glioma (H3 G34 mutant), diffuse high-grade glioma (H3 wild-type and IDH-wild-type), or infant-type hemispheric glioma.

In some embodiments, the glioma is a circumscribed astrocytic glioma. In some embodiments, the circumscribed astrocytic glioma is selected from pilocytic astrocytoma, high-grade astrocytoma with piloid features, pleomorphic xanthoastrocytoma, subependymal giant cell astrocytoma, chordoid glioma, or astroblastoma (MN1 altered).

In some embodiments, the glioma is progressive IDH1 or IDH-mutant, non-enhancing glioma. In some embodiments, the glioma is unmethylated MGMT glioma. In some embodiments, the glioma is unmethylated glioma.

In some embodiments, the human subject has recurrent high-grade glioma and DNA damage repair deficiency.

In some embodiments, the primary brain cancer is astrocytoma. In some embodiments, the metastatic brain cancer is astrocytoma. In some embodiments, the central nervous system cancer is astrocytoma. In some embodiments, the astrocytoma is recurrent astrocytoma. In some embodiments, the astrocytoma is newly diagnosed astrocytoma.

In some embodiments, the primary brain cancer is oligodendroglioma. In some embodiments, the metastatic brain cancer is oligodendroglioma. In some embodiments, the central nervous system cancer is oligodendroglioma. In some embodiments, the astrocytoma is recurrent oligodendroglioma. In some embodiments, the oligodendroglioma is newly diagnosed oligodendroglioma.

In some embodiments, the human subject demonstrates unbound concentrations of niraparib >5-fold of the biochemical IC50 value of niraparib in tumor tissue of the brain cancer. In some embodiments, the human subject demonstrates unbound concentrations of niraparib >5-fold of the biochemical IC50 value of niraparib in non-enhancing or enhancing tumor tissue of the brain cancer. In some embodiments, the human subject demonstrates unbound concentrations of niraparib >5-fold of the biochemical IC50 value of niraparib in non-enhancing tumor tissue of the brain cancer. In some embodiments, the human subject demonstrates unbound concentrations of niraparib >5-fold of the biochemical IC50 value of niraparib in enhancing tumor tissue of the brain cancer. In some embodiments, the unbound concentrations of niraparib in the in non-enhancing or enhancing tumor tissue of the brain cancer are measured after pre-surgical niraparib treatment. In some embodiments, the unbound concentrations of niraparib in tumor tissue are measured post-resection. In some embodiments, the unbound concentrations of niraparib are measured in brain tumor tissue samples collected intraoperatively. In some embodiments, the human subject demonstrates unbound concentrations of niraparib >5-fold of the biochemical IC50 value of niraparib within the gadolinium-nonenhancing region of the tumor of the brain cancer. In some embodiments, the unbound concentrations of niraparib within the gadolinium-nonenhancing region of the tumor of the brain cancer are measured after 4 days of pre-surgical niraparib (300 mg QD) treatment prior to planned resection at 3-5 or 8-12 hours following the last dose. In some embodiments, the unbound concentrations of niraparib within the gadolinium-nonenhancing region of the tumor of the brain cancer are measured after 4 days of pre-surgical niraparib (200 mg QD) treatment prior to planned resection at 3-5 or 8-12 hours following the last dose. In some embodiments, the unbound concentrations of niraparib are measured in brain tumor tissue samples collected intraoperatively.

In some embodiments, 5-fold of the biochemical IC50 value of niraparib is 19 nM. In some embodiments, 5-fold of the biochemical IC50 value of niraparib is about 19 nM.

In some embodiments, the brain/plasma ratio of niraparib is about 0.5. In some embodiments, the tumor/plasma ratio of niraparib is about 4. In some embodiments, the tumor/plasma ratio of niraparib is about 8. In some embodiments, the tumor/plasma ratio is measured in non-enhancing tumor tissue. In some embodiments, the tumor/plasma ratio is measured in enhancing tumor tissue. In some embodiments, the tumor/plasma ratio of niraparib is about 4 in non-enhancing tumor tissue. In some embodiments, the tumor/plasma ratio of niraparib is about 8 in enhancing tumor tissue.

In some embodiments, the niraparib, or pharmaceutically acceptable salt thereof, is administered in combination with one or more additional active agents known to be useful in the treatment of cancer.

In some embodiments, the one or more additional active agents is temozolomide, bevacizumab, or a combination thereof. In some embodiments, the one or more additional active agents comprises temozolomide, bevacizumab, pharmaceutically acceptable salts thereof, or combinations thereof.

In some embodiments, the one or more additional active agents is temozolomide.

In some embodiments, the one or more additional active agents is atezolizumab. In some embodiments, the one or more additional active agents comprises atezolizumab, or a pharmaceutically acceptable salt thereof.

In some embodiments, the one or more additional active agents is pembrolizumab. In some embodiments, the one or more additional active agents comprises pembrolizumab, or a pharmaceutically acceptable salt thereof.

In some embodiments, the one or more additional active agents is tovorafenib. In some embodiments, the one or more additional active agents comprises tovorafenib, or a pharmaceutically acceptable salt thereof.

In some embodiments, the one or more additional active agents is dostarlimab. In some embodiments, the one or more additional active agents comprises dostarlimab, or a pharmaceutically acceptable salt thereof.

In some embodiments, the human subject or cancer has a complete or partial response to platinum-based chemotherapy.

In some embodiments, the cancer is platinum insensitive.

In some embodiments, the cancer is platinum sensitive.

In some embodiments, the cancer is homologous recombination deficient (HRD) negative.

In some embodiments, the patient is characterized by having a deleterious or suspected deleterious mutation in BRCA1 and/or BRCA2.

In some embodiments, the primary or metastatic brain cancer is recurrent.

In some embodiments, the cancer is brain cancer.

In some embodiments, the brain cancer is primary brain cancer.

In some embodiments, the brain cancer is glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

In some embodiments, the niraparib administration begins after resection of the metastatic brain cancer tumor.

In some embodiments, the niraparib, or pharmaceutically acceptable salt thereof, is administered as maintenance therapy.

In some embodiments, the niraparib, or a pharmaceutically acceptable salt thereof, is administered in a dose that is equivalent to 200 mg or 300 mg of niraparib free base. In some embodiments, the niraparib, or a pharmaceutically acceptable salt thereof, is administered in a dose that is equivalent to about 300 mg of niraparib free base. In some embodiments, the niraparib, or a pharmaceutically acceptable salt thereof, is administered in a single daily dose. In some embodiments, the niraparib, or a pharmaceutically acceptable salt thereof, is administered twice per day. In some embodiments, the niraparib, or a pharmaceutically acceptable salt thereof, is administered in a daily dose that is equivalent to 200 mg or 300 mg of niraparib free base. In some embodiments, the niraparib, or a pharmaceutically acceptable salt thereof, is administered in a daily dose that is equivalent to about 300 mg of niraparib free base. In some embodiments, the niraparib, or a pharmaceutically acceptable salt thereof, is administered in a daily dose that is equivalent to about 200 mg of niraparib free base.

In some embodiments, the niraparib, or a pharmaceutically acceptable salt thereof, is dosed as niraparib tosylate monohydrate.

In some embodiments, the niraparib, or a pharmaceutically acceptable salt thereof, is administered in the form of a tablet.

In some embodiments, the dose of niraparib, or a pharmaceutically acceptable salt thereof, is administered daily.

In some embodiments, the primary recurrent brain cancer is selected from the group consisting of anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumour, pinealoma, diffuse intrinsic pontine glioma, IDH1/2(+) ATRX mutant glioma, malignant glioma and primitive neuroectodermal tumor of the brain.

In some embodiments, the primary recurrent brain cancer is a WHO grade II-IV tumor.

In some embodiments, the primary recurrent brain cancer is IDH1/2(+) ATRX mutant glioma.

In some embodiments, the cancer is brain cancer.

In some embodiments, the brain cancer is primary brain cancer.

In some embodiments, the brain cancer is glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

In some embodiments, the human subject demonstrates a chromosomal fusion with a cutoff Ct value of 35 in a C-circle assay. In some embodiments, the chromosomal fusion in the C-circle assay is measured after 4 days of pre-surgical niraparib (300 mg QD) treatment prior to planned resection at 3-5 or 8-12 hours following the last dose.

In some embodiments, the brain/plasma ratio of niraparib is about 0.5.

In some embodiments, the niraparib, or a pharmaceutically acceptable salt thereof, is administered in combination with one or more additional active agents known to be useful in the treatment of cancer.

In some embodiments, the one or more additional active agents is temozolomide, bevacizumab, or a combination thereof. In some embodiments, the one or more additional active agents comprises temozolomide, bevacizumab, pharmaceutically acceptable salts thereof, or combinations thereof.

In some embodiments, the one or more additional active agents is temozolomide. In some embodiments, the one or more additional active agents comprises temozolomide, or a pharmaceutically acceptable salt thereof.

In some embodiments, the one or more additional active agents is atezolizumab. In some embodiments, the one or more additional active agents comprises atezolizumab, or a pharmaceutically acceptable salt thereof.

In some embodiments, the one or more additional active agents is pembrolizumab. In some embodiments, the one or more additional active agents comprises pembrolizumab, or a pharmaceutically acceptable salt thereof.

In some embodiments, the one or more additional active agents is tovorafenib. In some embodiments, the one or more additional active agents comprises tovorafenib, or a pharmaceutically acceptable salt thereof.

In some embodiments, the one or more additional active agents is dostarlimab. In some embodiments, the one or more additional active agents comprises dostarlimab, or a pharmaceutically acceptable salt thereof.

In some embodiments, the human subject or cancer has a complete or partial response to platinum-based chemotherapy.

In some embodiments, the cancer is platinum insensitive.

In some embodiments, the cancer is platinum sensitive.

In some embodiments, the cancer is homologous recombination deficient (HRD) negative.

In some embodiments, the human subject or cancer is not tested for homologous recombination deficiency (HRD) status prior to administration of niraparib or a pharmaceutically acceptable salt thereof.

In some embodiments, the human subject or cancer is not tested for BRCA1 and/or BRCA2 mutation prior to administration of niraparib or a pharmaceutically acceptable salt thereof.

In some embodiments, the cancer is brain cancer.

In some embodiments, the brain cancer is primary brain cancer.

In some embodiments, the brain cancer is glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

In some embodiments, the metastatic brain cancer has spread from an original site in the lung, breast, colon, kidney and melanoma.

In some embodiments, the metastatic brain cancer is asymptomatic or active progressing brain metastases.

In some embodiments, the metastatic brain cancer is caused by a lung cancer selected from a solid tumor, squamous cell carcinoma of the lung, small cell lung cancer (SCLC), non-small cell lung cancer (NSCLC) and lung adenocarcinoma.

In some embodiments, the metastatic brain cancer is caused by a breast cancer selected from a solid tumor, ductal carcinoma in situ (DCIS; intraductal carcinoma), invasive breast cancer (ILC or IDC; invasive lobular carcinoma or invasive ductal carcinoma), triple negative breast cancer (TNBC), and inflammatory breast cancer.

In some embodiments, the metastatic brain cancer is caused by a kidney cancer selected from a solid tumor, kidney clear cell cancer, kidney papillary cancer, kidney chromophobe cancer, kidney renal cell carcinoma, urothelial carcinoma, kidney sarcoma, Wilms tumor, and kidney lymphoma.

In some embodiments, the metastatic brain cancer is caused by a colon cancer selected from colorectal cancer, squamous cell carcinoma, gastrointestinal neuroendocrine tumors, a solid tumor and adenocarcinoma.

In some embodiments, the metastatic brain cancer is caused by a melanoma selected from superficial spreading melanoma, nodular melanoma, lentigo maligna melanoma, acral lentiginous melanoma, choroidal melanoma, conjunctival melanoma, iris melanoma, and mucosal melanoma.

In some embodiments, the niraparib administration begins after resection of the metastatic brain cancer tumor.

In some embodiments, the niraparib, or a pharmaceutically acceptable salt thereof, is administered as maintenance therapy.

In some embodiments, the niraparib, or a pharmaceutically acceptable salt thereof, is administered in a dose that is equivalent to 200 mg or 300 mg of niraparib free base. In some embodiments, the niraparib, or a pharmaceutically acceptable salt thereof, is administered in a dose that is equivalent to about 300 mg of niraparib free base. In some embodiments, the niraparib, or a pharmaceutically acceptable salt thereof, is administered in a single daily dose. In some embodiments, the niraparib, or a pharmaceutically acceptable salt thereof, is administered twice per day. In some embodiments, the niraparib, or a pharmaceutically acceptable salt thereof, is administered in a daily dose that is equivalent to 200 mg or 300 mg of niraparib free base. In some embodiments, the niraparib, or a pharmaceutically acceptable salt thereof, is administered in a daily dose that is equivalent to about 300 mg of niraparib free base. In some embodiments, the niraparib, or a pharmaceutically acceptable salt thereof, is administered in a daily dose that is equivalent to about 200 mg of niraparib free base.

In some embodiments, the niraparib, or a pharmaceutically acceptable salt thereof, is dosed as niraparib tosylate monohydrate.

In some embodiments, the niraparib, or a pharmaceutically acceptable salt thereof, is administered in the form of a tablet.

In some embodiments, the dose of niraparib, or a pharmaceutically acceptable salt thereof, is administered daily.

In some embodiments, the brain/plasma ratio of niraparib is about 0.5.

In some embodiments, the niraparib, or a pharmaceutically acceptable salt thereof, is administered in combination with one or more additional active agents known to be useful in the treatment of cancer.

In some embodiments, the one or more additional active agents is temozolomide, bevacizumab, or a combination thereof. In some embodiments, the one or more additional active agents comprises temozolomide, bevacizumab, pharmaceutically acceptable salts thereof, or combinations thereof.

In some embodiments, the one or more additional active agents is temozolomide. In some embodiments, the one or more additional active agents comprises temozolomide, or a pharmaceutically acceptable salt thereof.

In some embodiments, the one or more additional active agents is atezolizumab. In some embodiments, the one or more additional active agents comprises atezolizumab, or a pharmaceutically acceptable salt thereof.

In some embodiments, the one or more additional active agents is pembrolizumab. In some embodiments, the one or more additional active agents comprises pembrolizumab, or a pharmaceutically acceptable salt thereof.

In some embodiments, the one or more additional active agents is tovorafenib. In some embodiments, the one or more additional active agents comprises tovorafenib, or a pharmaceutically acceptable salt thereof.

In some embodiments, the one or more additional active agents is dostarlimab. In some embodiments, the one or more additional active agents comprises dostarlimab, or a pharmaceutically acceptable salt thereof.

In some embodiments, the human subject or cancer has a complete or partial response to platinum-based chemotherapy.

In some embodiments, the cancer is platinum insensitive.

In some embodiments, the cancer is platinum sensitive.

In some embodiments, the cancer is homologous recombination deficient (HRD) negative.

In some embodiments, the cancer is brain cancer.

In some embodiments, the brain cancer is primary brain cancer.

In some embodiments, the brain cancer is glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

### I. Definitions

Reference throughout this specification to “one embodiment” or “an embodiment” or “some embodiments” or “a certain embodiment” means that a particular feature, structure, or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

As used herein, the term “administration” typically refers to the administration of a composition to a subject or system. Those of ordinary skill in the art will be aware of a variety of routes that may, in appropriate circumstances, be utilized for administration to a subject, for example a human subject. In some embodiments, administration may involve dosing that is intermittent (e.g., a plurality of doses separated in time) and/or periodic (e.g., individual doses separated by a common period of time) dosing.

As used herein, the terms “dosage form” or “unit dosage form” refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a regimen that has been determined to correlate with a desired or beneficial outcome when administered to a relevant population (i.e., with a therapeutic regimen). Those of ordinary skill in the art appreciate that the total amount of a therapeutic composition or agent administered to a particular subject is determined by one or more attending physicians and may involve administration of multiple dosage forms.

As used herein, the term “regimen” refers to a set of unit doses (typically more than one) that are administered individually to a subject, typically separated by one or more periods of time. In some embodiments, a given therapeutic agent is administered according to a regimen, which may involve one or more doses. In some embodiments, a regimen comprises a plurality of doses each of which is separated in time from other doses. In some embodiments, individual doses are separated from one another by a time period of the same length. In some embodiments, a regimen comprises a plurality of doses, wherein the doses are separated by time periods of different length. In some embodiments, a regimen comprises doses of the same amount. In some embodiments, a regimen comprises doses of different amounts. In some embodiments, a regimen comprises at least one dose, wherein the dose comprises one unit dose of the therapeutic agent. In some embodiments, a regimen comprises at least one dose, wherein the dose comprises two or more unit doses of the therapeutic agent. For example, a dose of 300 mg can be administered as a single 300 mg unit dose or as two 150 mg unit doses. In some embodiments, a regimen is correlated with or result in a desired or beneficial outcome when administered across a relevant population (i.e., is a therapeutic regimen). In some embodiments, the regimen comprises doses for the entire duration for a line of treatment to reach a desired result or beneficial outcome, or until disease progression or unacceptable adverse reaction is reached.

As used herein, the term “patient”, “subject”, or “test subject” refers to any organism to which provided compound or compounds described herein are administered in accordance with the present invention, e.g., for experimental, diagnostic, prophylactic, and/or therapeutic purposes. Typical subjects include animals (e.g., mammals such as mice, rats, rabbits, non-human primates, and humans; insects; worms; etc.). In a preferred embodiment, a subject is a human. In some embodiments, a subject may be suffering from, and/or susceptible to a disease, disorder, and/or condition (e.g., cancer such as brain cancer). In some embodiments, a patient is a human that has been diagnosed with a primary or metastatic brain cancer. In some embodiments, the patient has glioblastoma. In some embodiments, the patient has a WHO grade II-IV glioma. In some embodiments, the WHO grade II-IV glioma is recurrent. In some embodiments, the patient has IDH1/2(+) ATRX mutant glioma. In some embodiments, the IDH1/2(+) ATRX mutant glioma is recurrent. As used herein, a “patient population” or “population of subjects” refers to a plurality of patients or subjects.

As used herein, a “therapeutically effective amount” or “effective dose” refers to an amount of a therapeutic agent that produces the desired effect for which it is administered. In some embodiments, the term refers to an amount that is sufficient, when administered to a population suffering from or susceptible to a disease, disorder, and/or condition in accordance with a regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a regimen.

As used herein, a “chemotherapeutic agent” refers to a chemical agent that inhibits the proliferation, growth, life-span and/or metastatic activity of cancer cells. In some embodiments, a chemotherapeutic agent is platinum-based, e.g., a platinum agent. In some such embodiments, the platinum agent is selected from cisplatin, carboplatin, oxaliplatin, nedaplatin, triplatin tetranitrate, phenanthriplatin, picoplatin, or satraplatin.

As used herein, “homologous recombination” refers to a process wherein nucleotide sequences between distinct stands of DNA are exchanged. Homologous recombination is involved in a number of different biological processes, for example, homologous recombination occurs as part of the DNA repair process (e.g., doubled-strand break repair pathway and synthesis-dependent strand annealing pathway) and during process of meiosis/gametogenesis of eukaryotic organisms. As used herein, “homologous recombination deficiency,” “homologous recombination repair deficiency”, “homologous repair deficiency” or “HRD” refers to a reduction or impairment of the homologous recombination process. Such impairment may be via chromosomal aberrations or via mutations in one or more genes involved in DNA repair. Reduction or impairment of the homologous recombination process may also be measured by evaluating epigenetic alterations (i.e., hypermethylation) on the promoter of HRR genes, e.g., BRCA1 and RAD51C promoter methylation results in the repression of gene transcription and associated with PARP inhibitor sensitivity.

Reduction or impairment of the homologous recombination process may also be measured by a RAD51 foci formation assay: the absence of RAD51 foci formation demonstrates a defect in homologous recombination pathway.

Reduction or impairment of the homologous recombination process may also be measured by HRR, BRCA1, BRCA2 protein expression: abnormally lower protein levels of BRCA1, BRCA2, and other HRR genes may indicate a defect in homologous recombination pathway.

As used herein, “BRCA mutation” or “mutation of BRCA” refers to a change or difference in the sequence of at least one copy of either or both of the BRCA1 or BRCA2 genes relative to an appropriate reference sequence (e.g., a wild type reference and/or a sequence that is present in non-cancerous cells in the subject). A mutation in the BRCA1/2 gene may result in a BRCA1/2 deficiency, which may include, for example a loss or reduction in the expression or function of the BRCA gene and/or encoded protein. Such mutations may also be referred to as “deleterious mutations” or may be suspected to be deleterious mutations. A BRCA mutation can be a “germline BRCA mutation,” which indicates it was inherited from one or both parents. Germline mutations affect every cell in an organism and are passed on to offspring. A BRCA mutation can also be acquired during one's lifetime, i.e., spontaneously arising in any cell in the body (“soma”) at any time during the patient's life, (i.e., non-inherited), which is referred to herein as a “sporadic BRCA mutation” or a “somatic BRCA mutation” interchangeably. Genetic tests are available, and known by those of skill in the art.

As used herein, the term “genes involved in DNA repair” means any gene involved in repair of DNA in the cell. The components of the HR dependent DNA DSB repair pathway include, but are not limited to, ATM (NM-000051), RAD51 (NM-002875), RAD51LI (NM-002877), RAD51C (NM-002876), RAD51L3 (NM-002878), DMC1 (NM-007068), XRCC2 (NM7005431), XRCC3 (NM-005432), RAD52 (NM-002879), RAD54L (NM-003579), RAD54B (NM-012415), BRCA1 (NM-007295), BRCA2 (NM-000059), RAD50 (NM-005732), MRE11A (NM-005590), NBSI (NM-002485), ADPRT (PARP-1), ADPRTL2, (PARP2) CTPS, RPA, RPAI, RPA2, RPA3, XPD, ERCCI, XPF, MMS19, RAD51p, RAD51D, DMC1, XRCCR, XRCC3, RAD54, NB51, WRN, BLMKU70, RU80, ATR, CHKI, CHK2, FANCA, FANCB, FANCC, FANCD2, FANCE, FANCF, FANCG, RAD1, RAD9, BARD1 (NM_000465), PALB2 (NM_024675), BLM (NM_000057) and BRIP1 (NM_032043). Other proteins involved in the HR dependent DNA DSB repair pathway include regulatory factors such as EMSY (Cell (2003) 115:523-535).

One of skill in the art will be able to determine whether a gene is involved in DNA repair or homologous recombination. DNA repair status refers to the presence or absence of mutations in one or more of a gene involved in DNA repair. In some embodiments, the invention involves use of niraparib to treat a cancer patient regardless of DNA repair status.

As used herein, the term “progression free survival” means the time period for which a subject having a disease (e.g., cancer) survives, without a significant worsening of the disease state. Progression free survival may be assessed as a period of time in which there is no progression of tumor growth and/or wherein the disease status of a patient is not determined to be a progressive disease. In some embodiments, progression free survival of a subject having cancer is assessed by evaluating tumor (lesion) size, tumor (lesion) number, and/or metastasis.

As used herein, “progression free survival 2” (PFS2) is defined as time period from treatment randomization to the earlier date of assessment progression on the next anticancer therapy following study treatment or death by any cause. In some embodiments, determination of progression may be assessed by clinical and/or radiographic assessment.

The term “progression” of tumor growth or a “progressive disease” (PD) as used herein in reference to cancer status indicates an increase in the sum of the diameters of the target lesions (tumors). In some embodiments, progression of tumor growth refers to at least a 20% increase in the sum of diameters of target lesions, taking as reference the smallest sum on study (this includes the baseline sum if that is the smallest on study). In some embodiments, in addition to a relative increase of 20%, the sum of diameters of target lesions must also demonstrate an absolute increase of at least 5 mm. An appearance of one or more new lesions may also be factored into the determination of progression of tumor growth.

As used herein, the term “partial response” or “PR” refers to a decrease in tumor progression in a subject as indicated by a decrease in the sum of the diameters of the target lesions, taking as reference the baseline sum diameters. In some embodiments, PR refers to at least a 30% decrease in the sum of diameters or target lesions, taking as reference the baseline sum diameters. Exemplary methods for evaluating partial response are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

As used herein, “stabilization” of tumor growth or a “stable disease” (SD) refers to neither sufficient shrinkage to qualify for PR nor sufficient increase to qualify for PD. In some embodiments, stabilization refers to a less than 30%, 25%, 20%, 15%, 10% or 5% change (increase or decrease) in the sum of the diameters of the target lesions, taking as reference the baseline sum diameters. Exemplary methods for evaluating stabilization of tumor growth or a stable disease are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

As used herein, the term “complete response” or “CR” is used to mean the disappearance of all or substantially all target lesions. In some embodiments, CR refers to an 80%, 85%, 90%, 91%, 92%, 93%, 94%, 95%, 96%, 97%, 98%, 99% or 100% decrease in the sum of the diameters of the target lesions (i.e., loss of lesions), taking as reference the baseline sum diameters. In some embodiments, CR indicates that less than 10%, 9%, 8%, 7%, 6%, 5%, 4%, 3%, 2%, 1% or less of the total lesion diameter remains after treatment. Exemplary methods for evaluating complete response are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

As used herein, a “hazard ratio” (or “HR” when used in the context of niraparib treatment effect calculations, e.g., HR 0.38) is the expression of the hazard or chance of events occurring in the treatment arm as a ratio of the events occurring in the control arm. Hazard ratios may be determined by the Cox model, a regression method for survival data, which provides an estimate of the hazard ratio and its confidence interval. The hazard ratio is an estimate of the ratio of the hazard rate in the treated versus the control group. The hazard rate is the probability that if the event in question has not already occurred, it will occur in the next time interval, divided by the length of that interval. An assumption of proportional hazards regression is that the hazard ratio is constant over time.

As used herein, “HGG” refers to high grade glioma.

As used herein, “MGMT” is O-6-methylguanine-DNA methyltransferase, which is a gene that encodes a DNA repair enzyme. As used herein, “MGMT-unmethylated” or “unmethylated MGMT” refers to the absence of DNA methylation in the promoter region of the MGMT gene associated with a cancer, i.e., glioma or glioblastoma. As used herein, “hypermethylated MGMT promoter”, “MGMT promoter hypermethylation” or “methylated MGMT promoter” refers to refers to the presence of DNA methylation in the promoter region of the MGMT gene associated with a cancer, i.e., glioma or glioblastoma. As used herein, “MGMT status” refers to MGMT methylation status, i.e., positive status is associated with MGMT gene promoter methylation, and negative status is associated with unmethylated MGMT gene promoter.

As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. In some embodiments, “niraparib treatment” comprises administration of niraparib, or a pharmaceutically acceptable salt thereof, to a human subject in need thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue. A pharmaceutical composition can also refer to a medicament.

As used herein, the term “niraparib” means any of the free base compound ((3S)-3-[4-{7-(aminocarbonyl)-2H-indazol-2-yl}phenyl]piperidine), a salt form, including pharmaceutically acceptable salts, of (3S)-3-[4-{7-(aminocarbonyl)-2H-indazol-2-yl}phenyl]piperidine (e.g., (3S)-3-[4-{7-(aminocarbonyl)-2H-indazol-2-yl}phenyl]piperidine tosylate), or a solvated or hydrated form thereof (e.g., (3S)-3-[4-{7-(aminocarbonyl)-2H-indazol-2-yl}phenyl]piperidine tosylate monohydrate). In some embodiments, such forms may be individually referred to as “niraparib free base”, “niraparib tosylate” and “niraparib tosylate monohydrate”, respectively. Unless otherwise specified, the term “niraparib” includes all forms of the compound (3S)-3-[4-{7-(aminocarbonyl)-2H-indazol-2-yl}phenyl]piperidine.

As used herein, “intraoperatively” is defined as occurring during a surgical operation. In some embodiments, the surgical operation is resection of brain tumor tissue.

As used herein, “post-resection” refers to events taking place after resection of brain tumor tissue.

As used herein, “pre-surgical” or “presurgical” refers to events taking place prior to surgery. In some embodiments, the surgery is resection of brain tumor tissue. In some embodiments, “presurgical niraparib” or “pre-surgical niraparib” refers to administration of niraparib to a human subject prior to resection. For example, a patient identified as having brain cancer is administered niraparib for a period of time prior to resection, e.g., up to 7 days prior to resection, or for 4 days prior to resection.

As used herein, the term “maintenance therapy” or “maintenance treatment” is a treatment that is given to prevent relapse of a disease. For example, a maintenance therapy may prevent or minimize growth of a cancer after it has been substantially reduced or eliminated following an initial therapy (cancer treatment). Maintenance therapy may be a continuous treatment where multiple doses are administered at spaced intervals such as every day, every other day, every week, every 2 weeks, every 3 weeks, every 4 weeks, or every 6 weeks. In some embodiments, a maintenance therapy may continue for a predetermined length of time. In some embodiments, a maintenance therapy may continue until unacceptable toxicity occurs and/or disease progression occurs. In the course of maintenance treatment, treatment may be interrupted upon the occurrence of toxicity as indicated by an adverse event. If toxicity is appropriately resolved to baseline or grade 1 or less within 28 days, the patient may restart treatment with niraparib, which may include a dose level reduction, if prophylaxis is not considered feasible.

As used herein, overall survival (“OS”) is defined as time from commencement of treatment to death from any cause. With respect to use as a clinical trial endpoint, OS is defined as the time from randomization until death from any cause, and is measured in the intent to treat population.

As used herein, “objective response rate (“ORR”) is defined as the proportion of patients with tumor size reduction of a predefined amount and for a minimum period of time. Response duration is usually measured from the time of initial response until documented tumor progression. Generally, the ORR can be defined as the sum of partial responses plus complete responses.

As used herein, “time to first subsequent therapy” (TFST) is defined as the date of randomization in the current study to the start date of the first subsequent treatment regimen (e.g., anticancer therapy).

As used herein, “time to second subsequent therapy” (TSST) is defined as the date of randomization in the current study to the start date of the second subsequent treatment regimen (e.g., anticancer therapy).

As used herein, “chemotherapy-free interval” (CFI) is defined as the time from last dose of the last anticancer therapy (e.g., platinum-based chemotherapy) until the initiation of the next dose of anticancer therapy.

As used herein, “stereotactic radiation therapy”, is a highly focused radiation treatment that gives an intense dose of radiation concentrated on a tumor, while limiting the dose to the surrounding organs.

Niraparib is an orally available, selective poly(ADP-ribose) polymerase (PARP) 1 and 2 inhibitor. Niraparib has the following structure:

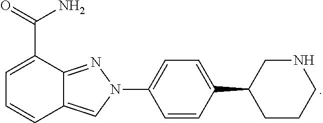 [SMILES: NC(=O)c1cccc2cn(-c3ccc([C@@H]4CCCNC4)cc3)nc12]

The chemical name for niraparib tosylate monohydrate is 2-{4-[(3S)-piperidin-3-yl]phenyl}-2H-indazole 7-carboxamide 4-methylbenzenesulfonate hydrate (1:1:1) and it has the following chemical structure:

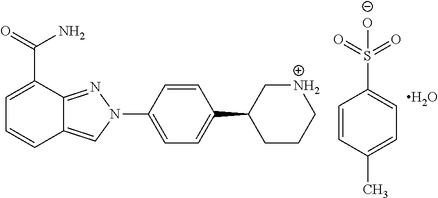 [SMILES: NC(=O)c1cccc2cn(-c3ccc([C@@H]4CCC[NH2+]C4)cc3)nc12 ; Cc1ccc(S(=O)(=O)[O-])cc1 ; O]

The empirical molecular formula for niraparib is C₂₆H₃₀N₄O₅S and its molecular weight is 510.61 g/mol. Niraparib tosylate monohydrate drug substance is a white to off-white, non-hygroscopic crystalline solid. Niraparib solubility is pH independent below the pKa of 9.95, with an aqueous free base solubility of 0.7 mg/mL to 1.1 mg/mL across the physiological pH range. Certain solid forms of niraparib are described in WO 2018/183354, which is incorporated by reference in its entirety.

Methods for preparation of niraparib include those described in WO 2014/088983; WO 2014/088984; WO 2018/200517, U.S. Pat. Nos. 8,071,623; 8,436,185; U.S. 62/489,415 filed Apr. 24, 2017; and Jones et al., J. Med. Chem., 52:7170-7185, 2009, each of which is incorporated by reference in its entirety.

Niraparib displays PARP 1 and 2 inhibition with IC₅₀=3.8 and 2.1 nM, respectively, and in a whole cell assay, it inhibited PARP activity with EC₅₀=4 nM and inhibited proliferation of cancer cells with mutant BRCA-1 and BRCA-2 with CC₅₀ in the 10-100 nM range (see Jones et al., Journal Medicinal Chemistry, 2009, 52, 7170-7185). Methods of administering niraparib to cancer patients are also described in WO2018/005818, which is hereby incorporated by reference in its entirety. Exemplary dosage forms comprising niraparib are described in, e.g., WO 2018/183349 and WO 2019/067634, each of which is incorporated by reference in its entirety.

### II. Combination Treatments

In an embodiment, the method of the invention may be used in combination with a further therapeutically active agent or agents known to be useful for the treatment of cancer (or in combination with one of more additional active agents), including immunotherapy (e.g., immune checkpoint inhibitor), cell and gene therapy, chemotherapy or radiation treatment. The term further therapeutically active agent or agents, as used herein, includes any compound or therapeutic agent known to or that demonstrates advantageous properties when administered to a patient in need of treatment for cancer. Furthermore, it does not matter if the compounds are administered in the same dosage form, e.g., one compound may be administered by injection and another compound may be administered orally. In some embodiments, the method of the invention is used as an add-on therapy to radiation therapy in the treatment of cancer. In some embodiments, niraparib, or a pharmaceutically acceptable salt thereof, is administered as an add-on therapy to radiation therapy in the treatment of brain cancer. In some embodiments, niraparib, or a pharmaceutically acceptable salt thereof, is administered as an add-on therapy to radiation therapy in the treatment of unmethylated MGMT glioma. In some embodiments, niraparib, or a pharmaceutically acceptable salt thereof, is administered as an add-on therapy to radiation therapy in the treatment of unmethylated MGMT glioblastoma. In some embodiments, niraparib, or a pharmaceutically acceptable salt thereof, is administered as an add-on therapy to radiation therapy in the treatment of unmethylated glioma. In some embodiments, niraparib, or a pharmaceutically acceptable salt thereof, is administered as an add-on therapy to radiation therapy in the treatment of unmethylated glioblastoma.

The term “co-administration” as used herein means either simultaneous administration or any manner of separate sequential administration of niraparib, or a pharmaceutically acceptable salt thereof, as described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g., one compound may be administered by injection and another compound may be administered orally.

Typically, any anti-neoplastic agent that has activity versus a susceptible tumor being treated may be co-administered in the treatment of cancer in the present invention. Co-administration is defined as including administration with a further agent or agents. Such further active agent or agents (or one or more additional active agents) may be selected from any known therapies for the treatment of cancer, including small molecules therapies, antibody therapies, antibody drug conjugate (ADC) therapies and cell & gene therapies. Examples of anti-neoplastic agents include, but are not limited to, chemotherapeutic agents, immune-modulators and immunostimulatory adjuvants. Examples of such agents can be found in Cancer Principles and Practice of Oncology by V. T. Devita, T. S. Lawrence, and S. A. Rosenberg (editors), 11^{th} edition (Nov. 29, 2018), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents; platinum coordination complexes; alkylating agents; antibiotic agents; topoisomerase I inhibitors; topoisomerase II inhibitors; antimetabolites; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents.

As used herein “immuno-modulators” refer to any substance including monoclonal antibodies that affects the immune system. The niraparib, or a pharmaceutical salt thereof, of the present invention can be considered immune-modulators. The niraparib, or a pharmaceutical salt thereof, of the present invention can be considered as an immuno-modulator. Immuno-modulators can be used as anti-neoplastic agents for the treatment of cancer. For example, immune-modulators include, but are not limited to, antibodies or other antagonists to CTLA-4, such as ipilimumab (YERVOY®) and tremelimumab; PD-1, such as dostarlimab, nivolumab (OPDIVO®), pembrolizumab (KEYTRUDA®), and cemiplimab (LIBTAYO®); and TIM-3, such as cobolimab. In some embodiments, the immune-modulator comprises ipilimumab, tremelimumab, dostarlimab, nivolumab, pembrolizumab, cemiplimab, cobolimab, or pharmaceutically acceptable salts thereof. Other immuno-modulators include, but are not limited to, antibodies or other antagonists to PD-L1, OX-40, LAG3, TIM-3, 41BB, and GITR.

In an embodiment, the further active agent (or additional active agent) could be selected from one as described in, for example WO 2018/208968, WO 2018/213732 and WO 2020/051142. For example, a PD-1 inhibitor such as dostarlimab or pembrolizumab. In some embodiments, the PD-1 inhibitor is dostarlimab. In some embodiments, the PD-1 inhibitor comprises dostarlimab, pembrolizumab, or pharmaceutically acceptable salts thereof. In some embodiments, the PD-1 inhibitor comprises dostarlimab, or a pharmaceutically acceptable salt thereof.

In some embodiments, the further active agent (or additional active agent) is a type II pan RAF-kinase inhibitor. Type II pan RAF-kinase inhibitors such as CCT3833/BAL3833, LY3009120, lifirafenib, belvarafenib, TAK-580, JZP815, and tovorafenib are being studied, e.g., in patients with solid tumors, advanced or metastasized tumors, advanced or refractory solid tumors, NRAS advanced melanoma, gliomas, colorectal cancer, primary brain tumors, brain metastases of solid tumors, malignant glioma, pediatric low-grade glioma (pLGG), and recurrent or progressive solid tumors. In some embodiments, the type II pan RAF-kinase inhibitor comprises CCT3833/BAL3833, LY3009120, lifirafenib, belvarafenib, TAK-580, JZP815, tovorafenib, or pharmaceutically acceptable salts thereof. In some embodiments, the type II pan RAF-kinase inhibitor is tovorafenib. In some embodiments, the type II pan RAF-kinase inhibitor comprises tovorafenib, or a pharmaceutically acceptable salt thereof.

In some embodiments, the active agent (or additional active agent) is an alkylating chemotherapeutic. In some embodiments, the alkylating chemotherapeutic is temozolomide. In some embodiments, the alkylating chemotherapeutic comprises temozolomide, or a pharmaceutically acceptable salt thereof. In some embodiments, temozolomide is administered to the human subject in combination with radiotherapy. In some embodiments, the human subject has positive MGMT status. In some embodiments, the human subject has negative MGMT status. In some embodiments, the temozolomide is administered to the human subject in combination with radiotherapy, wherein the human subject has positive MGMT status. In some embodiments, the temozolomide is administered to the human subject in combination with radiotherapy, wherein the human subject has negative MGMT status. In some embodiments, the human subject has newly diagnosed glioblastoma and MGMT promoter hypermethylation. In some embodiments, the human subject has unmethylated MGMT glioma. In some embodiments, the human subject has unmethylated MGMT glioblastoma. In some embodiments, the temozolomide is administered to the human subject in combination with radiotherapy, wherein the human subject has newly diagnosed glioblastoma and MGMT promoter hypermethylation. In some embodiments, treatment with an alkylating chemotherapeutic, for example temozolomide, is useful when the brain cancer is identified as having cells (particularly glioblastoma cells) with a methylated MGMT promoter. Therefore, in some embodiments, the temozolomide is administered to the human subject in combination with radiotherapy, wherein the human subject has a methylated MGMT promoter. In some embodiments, the temozolomide is administered as a pharmaceutically acceptable salt.

In some embodiments, the cancer is brain cancer.

In some embodiments, the brain cancer is primary brain cancer.

In some embodiments, the brain cancer is glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

In an embodiment, when the brain cancer is metastatic, the further active agent (or additional active agent) may be selected from the group consisting of a chemotherapy and immunotherapy, wherein the chemotherapy is selected from the group consisting of trastuzumab and erlotinib, and wherein the immunotherapy is selected from the group consisting of atezolizumab, ipilimumab, pembrolizumab and nivolumab. In some embodiments, the chemotherapy is tovorafenib. In some embodiments, the immunotherapy is pembrolizumab. In some embodiments, the immunotherapy is atezolizumab. In some embodiments, the immunotherapy is dostarlimab. In some embodiments, the chemotherapy is selected from the group consisting of trastuzumab, erlotinib and pharmaceutically acceptable salts thereof, and the immunotherapy is selected from the group consisting of atezolizumab, ipilimumab, pembrolizumab, nivolumab, and pharmaceutically acceptable salts thereof. In some embodiments, the chemotherapy comprises tovorafenib, or a pharmaceutically acceptable salt thereof. In some embodiments, the immunotherapy comprises pembrolizumab, or a pharmaceutically acceptable salt thereof. In some embodiments, the immunotherapy comprises atezolizumab, or a pharmaceutically acceptable salt thereof. In some embodiments, the immunotherapy comprises dostarlimab, or a pharmaceutically acceptable salt thereof.

In an embodiment, the further active agent (or additional active agent) may be selected from the group consisting of temozolomide and bevacizumab. In an embodiment, the further active agent (or additional active agent) comprises temozolomide, bevacizumab or pharmaceutically acceptable salts thereof.

In an embodiment, the further active agent (or additional active agent) may be temozolomide. In an embodiment, the further active agent (or additional active agent) comprises temozolomide, or a pharmaceutically acceptable salt thereof.

In an embodiment, the further active agent (or additional active agent) may be atezolizumab. In an embodiment, the further active agent (or additional active agent) comprises atezolizumab, or a pharmaceutically acceptable salt thereof.

In some embodiments, the further active agent (or additional active agent) may be pembrolizumab. In some embodiments, the further active agent (or additional active agent) comprises pembrolizumab, or a pharmaceutically acceptable salt thereof.

In some embodiments, the further active agent (or additional active agent) may be tovorafenib. In some embodiments, the further active agent (or additional active agent) comprises tovorafenib, or a pharmaceutically acceptable salt thereof.

In some embodiments, the further active agent (or additional active agent) may be dostarlimab. In some embodiments, the further active agent (or additional active agent) comprises dostarlimab, or a pharmaceutically acceptable salt thereof.

Select anti-neoplastic agents that may be used in combination with niraparib, or a pharmaceutically acceptable salt thereof, include but are not limited to: abarelix, abemaciclib, abiraterone, afatinib, aflibercept, aldoxorubicin, alectinib, alemtuzumab, arsenic trioxide, asparaginase, axitinib, AZD-9291, belinostat, bendamustine, bevacizumab, blinatumomab, bosutinib, brentuximab vedotin, cabazitaxel, cabozantinib, capecitabine, ceritinib, clofarabine, cobimetinib, crizotinib, daratumumab, dasatinib, degarelix, denosumab, dinutuximab, docetaxel, elotuzumab, entinostat, enzalutamide, epirubicin, eribulin, filgrastim, flumatinib, fulvestrant, fruquintinib, gemtuzumab ozogamicin, ibritumomab, ibrutinib, idelalisib, imatinib, irinotecan, ixabepilone, ixazomib, lenalidomide, lenvatinib, leucovorin, mechlorethamine, necitumumab, nelarabine, netupitant, nilotinib, obinutuzumab, olaparib, omacetaxine, osimertinib, oxaliplatin, paclitaxel, palbociclib, palonosetron, panitumumab, pegfilgrastim, peginterferon alfa-2b, pemetrexed, plerixafor, pomalidomide, ponatinib, pralatrexate, quizartinib, radium-223, ramucirumab, regorafenib, rolapitant, rucaparib, sipuleucel-T, sonidegib, sunitinib, talimogene laherparepvec, tipiracil, topotecan, trabectedin, trifluridine, triptorelin, uridine, vandetanib, velaparib, vemurafenib, venetoclax, vincristine, vismodegib, zoledronic acid, and pharmaceutically acceptable salts thereof.

### III. Brain Cancer and Study Rationale

The World Health Organization (WHO) has classified brain tumors into four categories based on increased severity, as summarized in Table A below.

TABLE A
Brain Cancer - WHO Grade

| | |
|---|---|
| GRADE I | Pilocytic Astrocytoma (also called |
| | Juvenile Pilocytic Astrocytoma) |
| GRADE II | Low-grade Astrocytoma |
| | Cytologic Atypia |
| | Variation in nuclear shape and size |
| | Hyperchromasia |
| GRADE III | Anaplastic Astrocytoma |
| | Anaplasia and increased mitotic activity |
| | Increased cellularity |
| GRADE IV | Glioblastoma (GBM) |
| | Microvascular proliferation and necrosis |

In glioblastoma (GBM), a non-limiting list of genes that may be altered or mutated is provided in Table B.

TABLE B
Exemplary list of genes that may be
altered or mutated in Glioblastoma

Isocitrate dehydrogenase (IDH)
O⁶-methylguanine-DNA methyltransferase (MGMT)
Epidermal growth factor receptor (EGFR)
Telomerase reverse transcriptase (TERT)
Chromosome 7p gain
Chromosome 10q loss
H3 histone, family 3A (H3F3A)
Fibroblast growth factor receptor (FGFR)
Neurotrophic tyrosine receptor kinase (NTRK)
α thalassemia/mental retardation syndrome X-linked (ATRX)

Primary brain tumors are among the top 10 causes of cancer-related deaths in the United States, accounting for approximately 1.4% of all cancers and 2.4% of all cancer-related deaths. About 14 per 100,000 people in the United States are diagnosed with a primary brain tumor each year, and 6 to 8 per 100,000 are diagnosed with a WHO grade III or IV primary brain tumor. Almost all grade II gliomas progress to high grade gliomas (Grade III/IV). Glioblastoma Multiforme (GBM, WHO Grade IV gliomas) is the most frequently reported malignant brain tumor histology (29.6%) in the National Cancer Database. The prognosis for patients who develop WHO Grade III or IV gliomas is bleak, with average survival after diagnosis ranging from 12-16 months. Although conventional treatment with surgery, irradiation, and temozolomide postpones tumor progression and extends patients survival, these tumors universally recur and unrelentingly result in patient death. Nevertheless, the alkylating agent temozolomide remains the only effective adjuvant chemotherapy available for glioblastoma patients. Phase 0 trials identify promising new drugs by ‘humanizing’ preclinical studies. An array of design variations exists under the Phase 0 umbrella to address a range of possible study objectives. These include studies to (1) determine whether a mechanism of action (MOA) defined in nonclinical models is achievable in humans; (2) refine a biomarker assay using human tumor tissue; (3) develop a novel imaging probe and evaluate its distribution, binding characteristics, and target effects in humans; (4) evaluate the human pharmacodynamics (PD) and/or pharmacokinetics (PK) of 2 or more analogs to select the most promising candidate for further development; (5) determine a dose-range and sequence of administration of a biomodulator for use in combination with established chemotherapy; and (6) provide human PK-PD relationship data for an agent before Phase 1 testing. For CNS oncology studies, PK analysis refers to measurement of study drug concentration in brain tumor tissue and PD analysis refers to quantification of a molecular/cellular target influenced by the study drug.

For brain tumor patients, Phase 0 clinical trials are challenging, not only due to trial logistics, but also because of the dampening effect the non-therapeutic nature of such studies has on patient accrual. A Phase 0 trial with an expansion phase adapts the Phase 0 strategy to brain tumor patients but incorporates a PK- and/or PD-dependent trigger that graduates Phase 0 patients into an exploratory expansion phase. In doing so, this tactic is compelling to potential brain tumor patients by providing them with the confidence that, if selected for treatment, there is biological evidence suggesting their tumor can respond. For these patients graduating to expansion phase, they (and their providers) are motivated by the biological rationale connecting the experimental therapy to their individual cases. Anecdotally, our institutional experience with both Phase 0 and Phase 0+expansion studies speak to this advantage and, since transitioning to the latter model in 2016, patient accrual has increased from 14% to 35% (Tien, et al, 2019).

Less than 1% of all published clinical trials for brain tumors contain both PK and PD endpoints evaluating tissue effects following initial drug exposure. Fewer studies, however, examine tissue from these same patients following extended periods of drug treatment, even though 19% of all high-grade glioma patients, for example, undergo 3 or more tumor resections. Using the proposed study paradigm, patients with planned re-resections for tumor recurrence following therapeutic dosing of the experimental agent(s) provide a critical opportunity for longitudinal tissue analysis. Within this population, enhancing and nonenhancing tumor tissue from fast- vs. slow-recurring tumors can be compared to identify the roles of on-target and off-target pathways in tumor escape. To control for interindividual variations in CNS drug penetration, putative resistance mechanisms can also be examined in matched tissue specimens from initial, second (Phase 0), and third (progressed from expansion phase) resections. Beyond characterizing resistance mechanisms, planned identification of tissue biomarker signatures associated with susceptibility to experimental agents can inform future clinical trial designs. For patients completing the Phase 0 component of the study with evidence of adequate tumor penetration (i.e., a ‘positive’ PK endpoint), variations in observed PD effects provide an opportunity to distinguish biological responders (i.e., patients with positive PK and PD endpoints) from non-responders (i.e., patients with a positive PK endpoint and negative PD endpoints). Using a variety of molecular and genetic techniques, a menu of tumor biomarker combinations predictive of pharmacodynamic sensitivity to the study drug(s) can be formulated for prospective interrogation. Taken together, these longitudinal studies of human brain tumors exposed to experimental therapies can provide actionable evidence for future strategies.

Thus, the Phase 0 clinical trial mechanism originally proposed by the FDA was conceived with the general drug development community in mind. Brain tumor drug development, however, poses unique study limitations due to the absence of predictive animal models, the significant risks of tumor acquisition, the unsuitability of microdosing, the challenge of the BBB, and the potentially confounding effects of neurosurgical anesthesia. Adapting the Phase 0 trial paradigm for neuro-oncology patients is an effective avenue to obtain direct evidence of drug delivery and target modulation. Specific modifications included in this proposal are: (1) abandoning microdosing in favor of a higher-dose regimen, (2) incorporating CSF into PK and PD analyses, and (3) adding an expansion component for patients with demonstrable PK and/or PD responses. In some embodiments, the patients in the expansion phase have primary brain cancer. In some embodiments, the patients in the expansion component have primary brain cancer. In some embodiments, the patients in the expansion phase have secondary brain cancer. In some embodiments, the patients in the expansion component have secondary brain cancer.

Primary brain cancer includes, but is not limited to those including anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumour, pinealoma, diffuse intrinsic pontine glioma, IDH1/2(+) ATRX mutant glioma, malignant glioma and primitive neuroectodermal tumor of the brain.

In some embodiments, the patient has glioblastoma (GBM). In some embodiments, one or more of the following genes is mutated or altered in the glioblastoma patient: Isocitrate dehydrogenase (IDH), O⁶-methylguanine-DNA methyltransferase (MGMT), Epidermal growth factor receptor (EGFR), Telomerase reverse transcriptase (TERT), Chromosome 7p gain, Chromosome 10q loss, H3 histone, family 3A (H3F3A), Fibroblast growth factor receptor (FGFR), Neurotrophic tyrosine receptor kinase (NTRK), or a thalassemia/mental retardation syndrome X-linked (ATRX). In some embodiments, the human subject has unmethylated MGMT glioblastoma. In some embodiments, the human subject has unmethylated glioblastoma.

Brain metastases (also known as secondary brain cancer) occur when cancer cells spread from the site of the primary cancer to the brain. Any cancer can spread to the brain, but the types most likely to cause brain metastases are lung, breast, colon, kidney and melanoma. Brain metastases may form one tumor or many tumors in the brain. In some embodiments, brain metastases are asymptomatic. In some embodiments, brain metastases are active progressing brain metastases.

In embodiments, the type of cancer causing brain metastases is a lung cancer (e.g., a solid tumor). In embodiments, a lung cancer is an advanced lung cancer. In embodiments, a lung cancer is a metastatic lung cancer. In embodiments, a lung cancer is squamous cell carcinoma of the lung. In embodiments, a lung cancer is small cell lung cancer (SCLC). In embodiments, a lung cancer is non-small cell lung cancer (NSCLC). In embodiments, a lung cancer is lung adenocarcinoma. In embodiments, a lung cancer is an ALK-translocated lung cancer (e.g., a lung cancer with a known ALK-translocation). In embodiments, a lung cancer is an EGFR-mutant lung cancer (e.g., a lung cancer with a known EGFR mutation). In embodiments, a lung cancer is a MSI-H lung cancer. In embodiments, a lung cancer is a MSS lung cancer. In embodiments, a lung cancer is a POLE-mutant lung cancer. In embodiments, a lung cancer is a POLD-mutant lung cancer. In embodiments, a lung cancer is a high TMB lung cancer. In embodiments, a lung cancer is associated with homologous recombination repair deficiency/homologous repair deficiency (“HRD”) or is characterized by a homologous recombination repair (HRR) gene mutation or deletion.

In embodiments, the type of cancer causing brain metastases is a breast cancer (e.g., a solid tumor). In embodiments, a breast cancer is ER-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or triple negative breast cancer (TNBC). In embodiments, a cancer is ductal carcinoma in situ (DCIS; intraductal carcinoma). In embodiments, a cancer is invasive breast cancer (e.g., ILC or IDC; invasive lobular carcinoma or invasive ductal carcinoma). In embodiments, a cancer is triple negative breast cancer (TNBC). In embodiments, a cancer is inflammatory breast cancer. In some embodiments, a breast cancer is a metastatic breast cancer. In some embodiments, a breast cancer is an advanced breast cancer. In some embodiments, a cancer is a stage II, stage III or stage IV breast cancer. In some embodiments, a cancer is a stage IV breast cancer. In some embodiments, a breast cancer is a triple negative breast cancer. In embodiments, a breast cancer is a metastatic breast cancer. In embodiments, a breast cancer is a MSI-H breast cancer. In embodiments, a breast cancer is a MSS breast cancer. In embodiments, a breast cancer is a POLE-mutant breast cancer. In embodiments, a breast cancer is a POLD-mutant breast cancer. In embodiments, a breast cancer is a high TMB breast cancer. In embodiments, a breast cancer is associated with homologous recombination repair deficiency/homologous repair deficiency (“HRD”) or is characterized by a homologous recombination repair (HRR) gene mutation or deletion.

In embodiments, the type of cancer causing brain metastases is a colon cancer (e.g., a solid tumor). In embodiments, a colon cancer is adenocarcinoma. In embodiments, the type of cancer causing brain metastases is a colorectal (CRC) cancer (e.g., a solid tumor). In embodiments, a colorectal cancer is an advanced colorectal cancer. In embodiments, a colorectal cancer is adenocarcinoma. In embodiments, a colorectal cancer is a metastatic colorectal cancer. In embodiments, a colorectal cancer is a MSI-H colorectal cancer. In embodiments, a colorectal cancer is a MSS colorectal cancer. In embodiments, a colorectal cancer is a POLE-mutant colorectal cancer. In embodiments, a colorectal cancer is a POLD-mutant colorectal cancer. In embodiments, a colorectal cancer is a high TMB colorectal cancer. In embodiments, a colorectal cancer is associated with homologous recombination repair deficiency/homologous repair deficiency (“HRD”) or is characterized by a homologous recombination repair (HRR) gene mutation or deletion. In embodiments, a colon cancer is selected from colorectal cancer, squamous cell carcinoma, gastrointestinal neuroendocrine tumors.

In embodiments, the type of cancer causing brain metastases is a kidney cancer (e.g., a solid tumor). In embodiments, a kidney cancer is kidney clear cell cancer. In embodiments, a kidney cancer is kidney papillary cancer. In embodiments, a kidney cancer is kidney chromophobe cancer. In embodiments, a kidney cancer is kidney renal cell carcinoma. In embodiments, a kidney cancer is urothelial carcinoma. In embodiments, a kidney cancer is kidney sarcoma. In embodiments, a kidney cancer is Wilms tumor. In embodiments, a kidney cancer is kidney lymphoma.

In embodiments, the type of cancer causing brain metastases is a melanoma. In embodiments, a melanoma is superficial spreading melanoma. In embodiments, a melanoma is nodular melanoma. In embodiments, a melanoma is lentigo maligna melanoma. In embodiments, a melanoma is acral lentiginous melanoma. In embodiments, a melanoma is choroidal melanoma. In embodiments, a melanoma is conjunctival melanoma. In embodiments, a melanoma is iris melanoma. In embodiments, a melanoma is mucosal melanoma. In embodiments, a melanoma is an advanced melanoma. In embodiments, a melanoma is a metastatic melanoma. In embodiments, a melanoma is a MSI-H melanoma. In embodiments, a melanoma is a MSS melanoma. In embodiments, a melanoma is a POLE-mutant melanoma. In embodiments, a melanoma is a POLD-mutant melanoma. In embodiments, a melanoma is a high TMB melanoma.

Exemplary DNA repair pathways and deficiencies therein are described in the present disclosure as well as in International Publication Nos. WO 2018/005818 and WO 2019/133697, each of which is incorporated herein by reference in its entirety. Exemplary DNA repair pathways include base excision repair (BER), direct repair (DR), double stranded break (DSB) repair, homologous recombination repair (HRR), mismatch repair (MMR), nucleotide excision repair (NER), and non-homologous end joining (NHEJ) repair; disruptions in these pathways can lead to the development and/or growth of cancer. See, e.g., Kelley et al., “Targeting DNA repair pathways for cancer treatment: what's new?”, Future Oncol., 10(7):1215-37 (2014).

In some embodiments, a patient receives PARP therapy (i.e., niraparib) independent of a deficiency in homologous repair in the cancer (HRD status). In some embodiments, a patient receives niraparib, or a pharmaceutically acceptable salt thereof, independent of HRD status. In some embodiments, the patient is not tested to determine the presence or absence of BRCA mutation or for the presence of homologous recombination deficiency, including for the presence of a deficiency in DNA repair, prior to administration of niraparib, or a pharmaceutically acceptable salt thereof.

In some embodiments, administration of niraparib, or a pharmaceutically acceptable salt thereof, commences within 12 weeks of the first day of the last cycle of chemotherapy.

In some embodiments, a patient receives niraparib, or a pharmaceutically acceptable salt thereof, independent of determination of HRD status (e.g., a patient receives niraparib, or a pharmaceutically acceptable salt thereof, independent of determination of BRCA status).

In some embodiments, the HRD status of a patient is known prior to therapy.

In some embodiments, the HRD status of a patient is determined. Various testing strategies have been utilized to measure the homologous recombination deficiency (HRD) in ovarian cancers (including fallopian and peritoneal cancers) and are known in the art. Genetic alterations of BRCA1/2 and other HRR-related genes can be sequenced to inform germline or somatic gene mutation status.

In some embodiments, a cancer is characterized by a deficiency in at least one, at least two, at least three, at least four, at least five, at least six, at least seven, at least eight, at least nine, at least ten, at least eleven, at least twelve, at least thirteen, at least fourteen, at least fifteen, or at least sixteen genes involved in the HRR pathway and which are not BRCA1 or BRCA2. In some embodiments, the at least one, at least two, at least three, at least four, at least five, at least six, at least seven, at least eight, at least nine, at least ten, at least eleven, at least twelve, at least thirteen, at least fourteen, or at least fifteen genes involved in the HRR pathway, and any combinations thereof.

In some embodiments, at least one deficiency in the HRR pathway is a mono-allelic mutation of a gene (e.g., a mono-allelic mutation of a gene that is not BRCA1 or BRCA2). In some embodiments, a mono-allelic mutation is independently a germline mutation. In some embodiments, a mono-allelic mutation is independently a sporadic mutation.

In some embodiments, at least one deficiency in the HRR pathway is a biallelic mutation of a gene (e.g., a bi-allelic mutation of a gene that is not BRCA1 or BRCA2). In some embodiments, a bi-allelic mutation is independently a germline mutation. In some embodiments, a bi-allelic mutation is independently a sporadic mutation.

Deficiencies in the HRR pathway (e.g., a deficiency in at least one non-BRCA1 or non-BRCA2 gene involved in the HRR pathway and/or a deficiency in BRCA1 and/or BRCA2) can be identified using methods known in the art. For example, the identification of a deficiency in the HRR pathway can include determinations made by a standardized laboratory test, such as and also including those tests approved by a relevant regulatory authority.

In some embodiments, a deficiency in a gene involved in the HRR pathway is identified using a pre-specified HRR gene panel. In some embodiments, a pre-specified HRR gene panel comprises one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L.

a. Surgical Status and Residual Disease

In some embodiments, a high risk patient has an inoperable cancer. In some embodiments, an inoperable cancer is a stage III cancer. In some embodiments, an inoperable cancer is a stage IV cancer.

In some embodiments, a platinum-based chemotherapy regimen also includes surgical treatment.

In some embodiments, surgical treatment occurs prior to commencement of the first line platinum-based chemotherapy regimen (primary debulking surgery).

In some embodiments, a high risk patient has residual disease following a debulking surgery. In some embodiments, residual disease is observed following primary debulking surgery.

In some embodiments, a surgery cannot completely remove cancer cells, and residual disease describes the cancer cells that remain post-surgery.

In some embodiments, residual disease is visible residual disease. In some embodiments, residual disease is less than about 2.0 cm. In some embodiments, residual disease is greater than about 0.1 cm. In some embodiments, residual disease is greater than about 0.1 cm and less than about 2.0 cm. In some embodiments, residual disease is greater than about 1.0 cm.

In some embodiments, a patient with stage III cancer has visible residual disease following surgery.

In some embodiments, a patient with stage IV cancer has visible residual disease following surgery.

b. Neoadjuvant Chemotherapy (NACT)

In some embodiments, a patient has received neoadjuvant chemotherapy (NACT), where a patient begins receiving platinum chemotherapy prior to surgical treatment, and the surgical treatment is an interval debulking surgery that occurs prior to completion of the platinum chemotherapy.

In embodiment, a patient receives two or more post-operative cycles of platinum-based therapy following interval debulking surgery.

In some embodiments, a patient with stage III cancer has received NACT. In some embodiments, a patient has no residual disease. In some embodiments, a patient has residual disease (e.g., visible residual disease as described herein).

In some embodiments, a patient with stage IV cancer has received NACT. In some embodiments, a patient has no residual disease. In some embodiments, a patient has residual disease (e.g., visible residual disease as described herein).

### 1. First Line Platinum-Based Chemotherapy.

In some embodiments, platinum-based chemotherapy is a regimen of cycles of treatment with a platinum agent (e.g., any platinum agent described herein). In some embodiments, administration of PARP therapy (e.g., niraparib) commences following completion of all cycles of treatment in a regimen. In some embodiments, a platinum-based treatment regimen is of about 3-6 months in duration. In some embodiments, patients must start niraparib treatment within 12 weeks of the first day of the last cycle of chemotherapy, e.g., within 11 weeks, within 10 weeks, within 9 weeks, within 8 weeks, within 7 weeks, within 6 weeks, within 5 weeks, within 4 weeks, within 3 weeks, within 2 weeks, or within 1 week of the first day of the last cycle of chemotherapy.

As used herein, a first line platinum-based chemotherapy regimen is the first regimen administered to a patient following a cancer diagnosis.

In some embodiments, a patient receives a platinum-based chemotherapy regimen comprising multiple cycles of platinum chemotherapy. A cycle of platinum chemotherapy can refer to a period of treatment with a platinum chemotherapy agent followed by a period of rest (no treatment) that is repeated on a regular schedule (thereby making up the platinum-based chemotherapy regimen).

In some embodiments, a platinum-based chemotherapy regimen comprises one, two, three, four, five, six, seven, eight, nine, ten, eleven, or twelve cycles of platinum chemotherapy. In some embodiments, a patient has received a platinum-based chemotherapy regimen of six or more cycles of platinum chemotherapy. In some embodiments, a patient has received a platinum-based chemotherapy regimen of nine or fewer cycles of platinum chemotherapy. In some embodiments, a patient has received a platinum-based chemotherapy regimen of 4-9 cycles of platinum chemotherapy; that is, a patient has received ≥4 and ≤9 cycles of platinum chemotherapy. In some embodiments, a patient has received a platinum-based chemotherapy regimen of 6-9 cycles of platinum chemotherapy; that is, a patient has received ≥6 and ≤9 cycles of platinum chemotherapy. In some embodiments, a patient has received a platinum-based chemotherapy regimen of 4-8 cycles of platinum chemotherapy; that is, a patient has received ≥4 and ≤8 cycles of platinum chemotherapy.

In some embodiments, a platinum-based chemotherapy regimen also includes surgical treatment. In some embodiments, debulking surgery occurs prior to administration of platinum chemotherapy to a patient described herein (primary debulking surgery). In some embodiments, a patient has received NACT, where a patient has received one or more cycles of platinum chemotherapy prior to a debulking surgery (interval debulking surgery). In some embodiments, a patient receiving NACT has received two or more (≥2) post-operative cycles of platinum chemotherapy following a debulking surgery.

Exemplary platinum chemotherapy agents suitable for methods described herein include cisplatin, carboplatin, oxaliplatin, nedaplatin, triplatin tetranitrate, phenanthriplatin, picoplatin, and/or satraplatin, or pharmaceutically acceptable salts thereof. In some embodiments, a first line platinum-based chemotherapy comprises administration of cisplatin or carboplatin, or pharmaceutically acceptable salts thereof.

In some embodiments, a first line platinum-based chemotherapy comprises administration of a second therapeutic agent in addition to a platinum chemotherapy agent.

In some embodiments, a first line platinum-based chemotherapy comprises administration of a second therapeutic agent that is a taxane chemotherapeutic (e.g., paclitaxel or docetaxel, or pharmaceutically acceptable salts thereof).

In some embodiments, a first line platinum-based chemotherapy comprises administration of a second therapeutic agent that is bevacizumab. In some embodiments, a first line platinum-based chemotherapy comprises administration of a second therapeutic agent that is bevacizumab, or a pharmaceutically acceptable salt thereof.

In some embodiments, a first line platinum-based chemotherapy is administered as intraperitoneal chemotherapy.

### 2. Response to First Line Platinum-Based Chemotherapy.

In some embodiments, administration of niraparib, or a pharmaceutically acceptable salt thereof, commences within 12 weeks of the first day of the last cycle of chemotherapy (e.g., platinum-based chemotherapy as described herein).

In some embodiments, a patient who receives PARP therapy (e.g., niraparib) has a cancer that has a complete response to the first line platinum-based chemotherapy regimen.

In some embodiments, a patient who receives PARP therapy (e.g., niraparib) has a cancer that has a partial response to the first line platinum-based chemotherapy regimen.

In some embodiments, a response (e.g., a complete response or a partial response) is assessed prior to completion of the first line platinum-based chemotherapy regimen. In some embodiments, a response (e.g., a complete response or a partial response) is assessed any time following the second or third cycle of platinum chemotherapy. In some embodiments, response is assessed after three or more (≥3) cycles of platinum chemotherapy.

Tumor response to either platinum-based chemotherapy or to the PARP therapy (e.g., niraparib administered following first line platinum-based chemotherapy) can be assessed according to methods known in the art.

Tumor response can be measured by, for example, by evaluating target and/or non-target lesions, including according to the RECIST v 1.1 guidelines, including as described herein. The guidelines are provided by E. A. Eisenhauer, et al., “New response evaluation criteria in solid tumors: Revised RECIST guideline (version 1.1.),” Eur. J. of Cancer, 45: 228-247 (2009), which is incorporated by reference in its entirety. The guidelines require, first, estimation of the overall tumor burden at baseline, which is used as a comparator for subsequent measurements. Tumors can be measured via use of any imaging system known in the art, for example, by a CT scan, or an X-ray. Measurable disease is defined by the presence of at least one measurable lesion. In studies where the primary endpoint is tumor progression (either time to progression or proportion with progression at a fixed date), the protocol must specify if entry is restricted to those with measurable disease or whether patients having non-measurable disease only are also eligible.

When more than one measurable lesion is present at baseline, all lesions up to a maximum of five lesions total (and a maximum of two lesions per organ) representative of all involved organs should be identified as target lesions and will be recorded and measured at baseline (this means in instances where patients have only one or two organ sites involved a maximum of two and four lesions respectively will be recorded).

Target lesions should be selected on the basis of their size (lesions with the longest diameter), be representative of all involved organs, but in addition should be those that lend themselves to reproducible repeated measurements.

Lymph nodes merit special mention since they are normal anatomical structures which may be visible by imaging even if not involved by tumor. Pathological nodes which are defined as measurable and may be identified as target lesions must meet the criterion of a short axis of P15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane; for MRI the plane of acquisition may be axial, sagittal or coronal). The smaller of these measures is the short axis.

For example, an abdominal node which is reported as being 20 mm 30 mm has a short axis of 20 mm and qualifies as a malignant, measurable node. In this example, 20 mm should be recorded as the node measurement. All other pathological nodes (those with short axis P10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as ‘present’, ‘absent’, or in rare cases ‘unequivocal progression.’ In addition, it is possible to record multiple nontarget lesions involving the same organ as a single item on the case record form (e.g., ‘multiple enlarged pelvic lymph nodes’ or ‘multiple liver metastases’).

The response of target lesions can be evaluated as follows according to response criteria by RECIST v 1.1:
Complete Response (CR): Disappearance of all target lesions. Any pathological lymph nodes (whether target or non-target) must have reduction in short axis to <10 mm.
Partial Response (PR): At least a 30% decrease in the sum of the diameters of target lesions, taking as reference the baseline sum diameters
Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progressions).
Stable Disease (SD): Neither sufficient shrinkage to qualify for PR nor sufficient increase to qualify for PD, taking as reference the smallest sum diameters while on study.

The response of non-target lesions can be evaluated as follows according to response criteria by RECIST v 1.1:
Complete Response (CR): Disappearance of all non-target lesions and normalization of tumor marker level. All lymph nodes must be non-pathological in size (<10 mm short axis)
Non-CR Non-PD: Persistence of one or more non-target lesion(s) and/or maintenance of tumor marker level above the normal limits.
Progressive Disease (PD): Appearance of one or more new lesions and/or unequivocal progression of existing non-target lesions. Unequivocal progression should not normally trump target lesion status. It must be representative of overall disease status change, not a single lesion increase.

### 3. Platinum Sensitive and Platinum Resistant Cancer

A platinum sensitive cancer is a cancer that has no relapse or disease progression for a minimum duration of about six months following treatment with a platinum chemotherapy.

A platinum resistant cancer is a cancer that has an initial response to treatment with platinum chemotherapy but relapse or disease progression was observed within about six months following treatment.

In some embodiments, a patient who receives niraparib receives therapy independent of the platinum sensitive status of the cancer.

In some embodiments, a patient who receives niraparib receives therapy independent of determining the platinum sensitive status of the cancer.

In some embodiments, a patient who receives niraparib has a cancer that is platinum sensitive.

In some embodiments, a patient who receives niraparib has a cancer that is platinum resistant.

### IV. General Protocol

As described herein, provided methods comprise administering niraparib, or a pharmaceutically acceptable salt thereof, to a patient, a subject, or a population of subjects according to a regimen that achieves any one of or combination of: prolonged progression free survival; reduced hazard ratio for disease progression or death; and/or prolonged overall survival or a positive overall response rate.

In some embodiments, methods described herein can be effective as first line maintenance therapy for brain cancer for patients who have received and completed only one platinum-based chemotherapy regimen.

In some embodiments, a method is suitable for prolonging the effect of first line platinum-based chemotherapy in a patient diagnosed with a cancer (e.g., brain cancer).

In some embodiments, the cancer is brain cancer.

In some embodiments, the brain cancer is primary brain cancer.

In some embodiments, the brain cancer is glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

In some embodiments, a method is suitable for treating primary or metastatic brain cancer.

In some embodiments, a suitable patient is newly diagnosed with primary or metastatic brain cancer. In some embodiments, a patient who is newly diagnosed with primary or metastatic brain cancer has not yet received any therapy for the cancer (e.g., a patient newly diagnosed with primary or metastatic brain cancer has not yet received any platinum chemotherapy, radiation therapy or any surgery). In some embodiments, a method comprises administering to said patient only one regimen of platinum-based chemotherapy. In some embodiments, a patient's cancer responds to the chemotherapy as evidenced by a complete or partial response. In some embodiments, a method comprises orally administering an effective dose of niraparib to said patient daily prior to a determination of said cancer's sensitivity to the regimen of platinum-based chemotherapy. In some embodiments, a cancer is platinum sensitive. In some embodiments, a cancer is platinum resistant.

In some embodiments, a suitable patient who has been diagnosed with primary or metastatic brain cancer has previously received and completed one regimen of platinum-based chemotherapy. For example, a suitable patient who has been diagnosed with primary or metastatic brain cancer has previously received and completed only one regimen of platinum-based chemotherapy.

In some embodiments, a suitable patient is a patient with a primary or metastatic brain cancer that is at high risk for progression of disease.

In some embodiments, a primary or metastatic brain cancer is characterized as having a BRCA deficiency and/or HRD (e.g., a positive HRD status).

In some embodiments, a primary or metastatic brain cancer is characterized by the absence of a germline BRCA mutation that is deleterious or suspected to be deleterious.

In some embodiments, a primary or metastatic brain cancer is characterized by the absence of a BRCA mutation either germline or sporadic.

In some embodiments, niraparib, or a pharmaceutically acceptable salt thereof, is administered simultaneously or sequentially with an additional therapeutic agent, such as, for example, a chemotherapeutic agent. In some embodiments, niraparib, or a pharmaceutically acceptable salt thereof, is administered before, during, or after administration of a chemotherapeutic agent. Administration of niraparib, or a pharmaceutically acceptable salt thereof, simultaneously or sequentially with an additional therapeutic agent (e.g., a chemotherapeutic agent) is referred to as “combination therapy.” In combination therapy, niraparib, or a pharmaceutically acceptable salt thereof, can be administered prior to (e.g., 5 minutes, 15 minutes, 30 minutes, 45 minutes, 1 hour, 2 hours, 4 hours, 6 hours, 12 hours, 24 hours, 48, hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the chemotherapeutic agent to a subject in need thereof. In some embodiments, niraparib, or a pharmaceutically acceptable salt thereof, and the chemotherapeutic agent are administered 1 minute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart.

In some embodiments, niraparib, or a pharmaceutically acceptable salt thereof, is administered to a patient or population of subjects who has exhibited response to a first line platinum-based chemotherapy regimen.

In some embodiments, niraparib, or a pharmaceutically acceptable salt thereof, is administered as a first line maintenance therapy to a patient following complete or partial response to a first line platinum-based chemotherapy regimen. Methods described herein are suitable for treating a patient diagnosed with a cancer that is primary or metastatic brain cancer and for prolonging the effect of a first line platinum-based chemotherapy regimen (e.g., as described herein).

In some embodiments, administration of niraparib, or a pharmaceutically acceptable salt thereof, commences within 12 weeks of the first day of the last cycle of chemotherapy (e.g., platinum-based chemotherapy as described herein).

In some embodiments, a PARP therapy (e.g., as administered following a first line platinum-based chemotherapy regimen) comprises at least one oral dose of niraparib, or a pharmaceutically acceptable salt thereof. In some embodiments, the regimen comprises a plurality of oral doses. In some embodiments, the regimen comprises once daily (QD) dosing.

In some embodiments, the PARP therapy regimen comprises at least one 28 day cycle. In some embodiments, the regimen comprises a plurality of 28 day cycles. In some embodiments, the regimen comprises one 28 day cycle. In some embodiments, the regimen comprises two 28 day cycles. In some embodiments, the regimen comprises three 28 day cycles. In some embodiments, the regimen comprises continuous 28 day cycles. In some embodiments, the regimen comprises administration of an effective dose of niraparib, or a pharmaceutically acceptable salt thereof, daily until disease progression or unacceptable toxicity occurs. In some embodiments, the regimen comprises a daily dose of at least 100, 200 or 300 mg niraparib, or a pharmaceutically acceptable salt thereof, per day dosed until disease progression or unacceptable toxicity occurs.

In some embodiments, the oral dose is an amount of niraparib within a range of about 5 to about 400 mg. In some embodiments, the amount of niraparib is about 5, about 10, about 25, about 50, about 100, about 150, about 200, about 250, about 300, about 350, or about 400 mg.

In some embodiments, the amount of niraparib is about 100 mg of niraparib. In some embodiments, the regimen comprises administration of 300 mg of niraparib once daily.

In some embodiments, the amount of niraparib is about 100 mg of niraparib. In some embodiments, the regimen comprises administration of 200 mg of niraparib once daily.

In some embodiments, the amount of niraparib is about 300 mg of niraparib. In some embodiments, the regimen comprises administration of 300 mg of niraparib once daily.

In some embodiments, a starting dose of niraparib treatment is based upon the patient's baseline body weight or baseline platelet count. In some embodiments, a patient with a baseline body weight ≥77 kg and baseline platelet count ≥150,000 μL is administered niraparib in an amount corresponding to about 300 mg niraparib freebase daily. In some embodiments, a patient with a baseline body weight <77 kg or baseline platelet count <150,000 μL is administered niraparib in an amount corresponding to about 200 mg niraparib freebase daily. Additional dose modifications of study treatment will not be based upon changes in the patient's body weight during study participation. For those patients whose starting dose is ˜200 mg niraparib once daily, escalation to ˜300 mg niraparib once daily is permitted if no treatment interruption or discontinuation occurred during the first 2 cycles of therapy. For any dose modification, the number of unit dosage forms (e.g., capsules or tablets) administered will be modified accordingly.

In some embodiments, the oral dose is administered in one or more unit dosage forms. In some embodiments, the one or more unit dosage forms are capsules. In some embodiments, the one or more unit dosage forms are tablets. In some embodiments, each unit dosage form comprises about 5, about 10, about 25, about 50, or about 100 mg of niraparib. It is understood that any combination of unit dosage forms can be combined to form a once daily (QD) dose. For example, three 100 mg unit dosage forms can be taken once daily such that about 300 mg of niraparib is administered once daily. In some embodiments, niraparib is administered as a single 300 mg unit dosage form. In some embodiments, niraparib is administered 300 mg QD. In some embodiments, niraparib is administered as 3×100 mg QD (i.e., niraparib is administered as three unit dosage forms of 100 mg). In some embodiments, niraparib is administered as 2×150 mg QD (i.e., niraparib is administered as two unit dosage forms of 150 mg).

In some embodiments, the niraparib is dosed as the free base form of niraparib. In some embodiments, the niraparib is dosed as a pharmaceutically acceptable salt of niraparib. In some embodiments, the niraparib is dosed as niraparib tosylate monohydrate. In some embodiments, the amount of niraparib in the dose is based on the weight of the free base of the pharmaceutically acceptable salt. In some embodiments, the amount of niraparib in the dose is based on the weight of the pharmaceutically acceptable salt.

In some embodiments, the oral dose of niraparib is administered at a 7 days on, 7 days off regimen. In some embodiments, the 7 days on, 7 days off regimen comprises 3 or more cycles. In some embodiments, the 7 days on, 7 days off regimen comprises 4 cycles. In some embodiments, the oral dose of niraparib is 45 mg/kg. In some embodiments, the oral dose of niraparib is administered in combination with one or more additional active agents known to be useful in the treatment of cancer. In some embodiments, the oral dose of niraparib is administered in combination with one additional active agent known to be useful in the treatment of cancer. In some embodiments, the oral dose of niraparib and the one additional active agent are each administered at a 7 days on, 7 days off regimen. In some embodiments, the 7 days on, 7 days off regimen comprises 3 or more cycles. In some embodiments, the 7 days on, 7 days off regimen comprises 4 cycles. In some embodiments, the niraparib is administered at the first, third, fifth, and seventh week of the regimen, and the one additional active agent is administered at the second, fourth, sixth, and eighth week of the regimen. In some embodiments, the oral dose of niraparib is 45 mg/kg and the oral dose of the one additional active agent is 60 mg/kg. In some embodiments, the one or more additional active agents is temozolomide, bevacizumab, or a combination thereof. In some embodiments, the one or more additional active agents comprises temozolomide, bevacizumab, pharmaceutically acceptable salts, or combinations thereof. In some embodiments, the one or more additional active agents is temozolomide. In some embodiments, the one or more additional active agents comprises temozolomide, or a pharmaceutically acceptable salt thereof. In some embodiments, the one or more additional active agents is atezolizumab. In some embodiments, the one or more additional active agents comprises atezolizumab, or a pharmaceutically acceptable salt thereof. In some embodiments, the one or more additional active agents is pembrolizumab. In some embodiments, the one or more additional active agents comprises pembrolizumab, or a pharmaceutically acceptable salt thereof. In some embodiments, the one or more additional active agents is tovorafenib. In some embodiments, the one or more additional active agents comprises tovorafenib, or a pharmaceutically acceptable salt thereof. In some embodiments, the one or more additional active agents is dostarlimab. In some embodiments, the one or more additional active agents comprises dostarlimab, or a pharmaceutically acceptable salt thereof.

In some embodiments, dose interruption (no longer than 28 days) or dose reduction can be allowed based on treatment side effects. For patients whose starting dose is about 300 mg niraparib daily, dose reductions to about 200 mg niraparib daily and subsequently to about 100 mg niraparib daily will be allowed.

In some embodiments, niraparib, or a pharmaceutically acceptable salt thereof, is administered for at least about 6 months. In some embodiments, niraparib, or a pharmaceutically acceptable salt thereof, is administered for at least about 9 months. In some embodiments, niraparib, or a pharmaceutically acceptable salt thereof, is administered for at least about 12 months. In some embodiments, niraparib, or a pharmaceutically acceptable salt thereof, is administered for at least about 15 months. In some embodiments, niraparib, or a pharmaceutically acceptable salt thereof, is administered for at least about 18 months. In some embodiments, niraparib, or a pharmaceutically acceptable salt thereof, is administered for at least about 21 months. In some embodiments, niraparib, or a pharmaceutically acceptable salt thereof, is administered for at least about 24 months. In some embodiments, niraparib, or a pharmaceutically acceptable salt thereof, is administered for at least about 27 months. In some embodiments, niraparib, or a pharmaceutically acceptable salt thereof, is administered for at least about 30 months. In some embodiments, niraparib, or a pharmaceutically acceptable salt thereof, is administered for at least about 33 months. In some embodiments, niraparib, or a pharmaceutically acceptable salt thereof, is administered for at least about 36 months. In some embodiments, niraparib, or a pharmaceutically acceptable salt thereof, is administered for at least about 39 months. In some embodiments, niraparib, or a pharmaceutically acceptable salt thereof, is administered for at least about 42 months. In some embodiments, niraparib, or a pharmaceutically acceptable salt thereof, is administered for at least about 45 months. In some embodiments, niraparib, or a pharmaceutically acceptable salt thereof, is administered for at least about 48 months.

In some embodiments, niraparib, or a pharmaceutically acceptable salt thereof, is administered for at least about 9-12 months. In some embodiments, niraparib, or a pharmaceutically acceptable salt thereof, is administered for at least about 12-15 months. In some embodiments, niraparib, or a pharmaceutically acceptable salt thereof, is administered for at least about 15-18 months. In some embodiments, niraparib, or a pharmaceutically acceptable salt thereof, is administered for at least about 18-21 months. In some embodiments, niraparib, or a pharmaceutically acceptable salt thereof, is administered for at least about 21-24 months. In some embodiments, niraparib, or a pharmaceutically acceptable salt thereof, is administered for at least about 24-27 months. In some embodiments, niraparib, or a pharmaceutically acceptable salt thereof, is administered for at least about 27-30 months. In some embodiments, niraparib, or a pharmaceutically acceptable salt thereof, is administered for at least about 30-33 months. In some embodiments, niraparib, or a pharmaceutically acceptable salt thereof, is administered for at least about 33-36 months. In some embodiments, niraparib, or a pharmaceutically acceptable salt thereof, is administered for at least about 36-39 months. In some embodiments, niraparib, or a pharmaceutically acceptable salt thereof, is administered for at least about 39-42 months. In some embodiments, niraparib, or a pharmaceutically acceptable salt thereof, is administered for at least about 42-45 months. In some embodiments, niraparib, or a pharmaceutically acceptable salt thereof, is administered for at least about 45-48 months.

In some embodiments, the methods prolong progression free survival as compared to control. In some embodiments, the methods reduce the hazard ratio for disease progression or death as compared to control. In some embodiments, the methods prolong overall survival as compared to control. In some embodiments, the methods achieve an overall response rate of at least 30%. In some embodiments, the methods achieve improved progression free survival 2 as compared to control. In some embodiments, the methods achieve improved chemotherapy free interval as compared to control. In some embodiments, the methods achieve improved time to first subsequent therapy as compared to control. In some embodiments, the methods achieve improved time to second subsequent therapy as compared to control. In some embodiments, the methods have been determined to not have a detrimental effect on Quality of Life as determined by FOSI and/or EQ-5D-5L. In some embodiments, the methods have been determined to not impact the effectiveness of a subsequent treatment with a chemotherapeutic agent (e.g., a platinum agent, including but not limited to, cisplatin, carboplatin, oxaliplatin, nedaplatin, triplatin tetranitrate, phenanthriplatin, picoplatin, or satraplatin).

Such a prolongation of progression free survival may result in a reduced hazard ratio for disease progression or death. Maintenance therapy is administered during the interval between cessation of a first line platinum-based chemotherapy regimen or radiation therapy with the goal of delaying disease progression and the subsequent intensive therapies that may present tolerability issues for patients. In another embodiment, the patients with primary or metastatic brain cancer are further characterized as having a BRCA deficiency or HRD. In some embodiments, the patient has glioblastoma. In some embodiments, the patient has a WHO grade II-IV glioma. In some embodiments, the patient has IDH1/2(+) ATRX mutant glioma.

In some embodiments, niraparib, or a pharmaceutically acceptable salt thereof, is administered as a maintenance therapy in patients with primary or metastatic brain cancer who have a complete response or partial response following administration and completion of only one platinum-based chemotherapy treatment, wherein said administration of niraparib, or a pharmaceutically acceptable salt thereof, results in prolongation of progression free survival. Such a prolongation of progression free survival may result in a reduced hazard ratio for disease progression or death. Such first line maintenance therapy is administered during the interval between cessation of chemotherapy with the goal of delaying disease progression and the subsequent intensive therapies that may present tolerability issues for patients. In some embodiments, the patients with primary or metastatic brain cancer are further characterized as having a BRCA deficiency or HRD. In some embodiments, the patients with primary or metastatic brain cancer are further characterized by the absence of a germline BRCA mutation that is deleterious or suspected to be deleterious. In some embodiments, the patient has glioblastoma. In some embodiments, the patient has a WHO grade II-IV glioma. In some embodiments, the patient has IDH1/2(+) ATRX mutant glioma.

In some embodiments, the present invention provides a method of administering niraparib, or a pharmaceutically acceptable salt thereof, to a patient having primary or metastatic brain cancer independent of the cancer's sensitivity to platinum. In some embodiments, the method comprises administering niraparib, or a pharmaceutically acceptable salt thereof, according to a regimen determined to achieve prolonged progression free survival. In some embodiments, the progression free survival is greater in patients receiving niraparib, or a pharmaceutically acceptable salt thereof, for example as compared with patients not receiving niraparib, or a pharmaceutically acceptable salt thereof. In some embodiments, progression free survival is greater in patients receiving niraparib, or a pharmaceutically acceptable salt thereof, than in patients receiving alternative cancer therapy, for example such as therapy with a different PARP inhibitor. In some embodiments, a patient has a primary or metastatic brain cancer that demonstrated a partial response to platinum-based chemotherapy prior to administering of niraparib, or a pharmaceutically acceptable salt thereof (e.g., a platinum sensitive or a platinum resistant primary or metastatic brain cancer that demonstrated a partial response to platinum chemotherapy). In some embodiments, a patient has a primary or metastatic brain cancer that demonstrated a complete response to platinum-based chemotherapy prior to administering of niraparib, or a pharmaceutically acceptable salt thereof (e.g., a platinum sensitive or a platinum resistant primary or metastatic brain cancer that demonstrated a complete response to platinum chemotherapy).

In some embodiments, the present invention provides methods of administering niraparib, or a pharmaceutically acceptable salt thereof, to a patient having platinum sensitive primary or metastatic brain cancer comprising administering niraparib, or a pharmaceutically acceptable salt thereof, according to a regimen determined to achieve prolonged progression free survival. In some embodiments, the progression free survival is greater in patients receiving niraparib, or a pharmaceutically acceptable salt thereof, for example as compared with patients not receiving niraparib, or a pharmaceutically acceptable salt thereof. In some embodiments, progression free survival is greater in patients receiving niraparib, or a pharmaceutically acceptable salt thereof, than in patients receiving alternative cancer therapy, for example such as therapy with a different PARP inhibitor. In some embodiments, a patient has a primary or metastatic brain cancer that demonstrated a partial response to platinum-based chemotherapy prior to administering of niraparib, or a pharmaceutically acceptable salt thereof. In some embodiments, a patient has a primary or metastatic brain cancer that demonstrated a complete response to platinum-based chemotherapy prior to administering of niraparib, or a pharmaceutically acceptable salt thereof.

In some embodiments, the present invention provides methods of administering niraparib, or a pharmaceutically acceptable salt thereof, to a patient having platinum resistant primary or metastatic brain cancer comprising administering niraparib, or a pharmaceutically acceptable salt thereof, according to a regimen determined to achieve prolonged progression free survival. In some embodiments, the progression free survival is greater in patients receiving niraparib, or a pharmaceutically acceptable salt thereof, for example as compared with patients not receiving niraparib, or a pharmaceutically acceptable salt thereof. In some embodiments, progression free survival is greater in patients receiving niraparib, or a pharmaceutically acceptable salt thereof, than in patients receiving alternative cancer therapy, for example such as therapy with a different PARP inhibitor. In some embodiments, a patient has a primary or metastatic brain cancer that demonstrated a partial response to platinum-based chemotherapy prior to administering of niraparib, or a pharmaceutically acceptable salt thereof. In some embodiments, a patient has a primary or metastatic brain cancer that demonstrated a complete response to platinum-based chemotherapy prior to administering of niraparib, or a pharmaceutically acceptable salt thereof.

In some embodiments, progression for the purposes of determining progression free survival is determined by 1) tumor assessment by CT/MRI showing unequivocal progressive disease according to RECIST 1.1 criteria; and/or 2) additional diagnostic tests (e.g., histology/cytology, ultrasound techniques, endoscopy, positron emission tomography) identifying new lesions.

In some embodiments, the patient is characterized by having homologous recombination deficiency. In some embodiments, the patient has a positive homologous recombination deficiency status. Homologous recombination deficiency status may be established according to methods known by those in the art.

In some embodiments, the prolonged progression free survival is at least about 6 months. In some embodiments, the prolonged progression free survival is at least about 9 months. In some embodiments, the progression free survival is at least about 12 months. In some embodiments, the progression free survival is at least about 15 months. In some embodiments, the progression free survival is at least about 18 months. In some embodiments, the progression free survival is at least about 21 months. In some embodiments, the progression free survival is at least about 24 months. In some embodiments, the progression free survival is at least about 27 months. In some embodiments, the progression free survival is at least about 30 months. In some embodiments, the progression free survival is at least about 33 months. In some embodiments, the progression free survival is at least about 36 months. In some embodiments, the progression free survival is at least about 39 months. In some embodiments, the progression free survival is at least about 42 months. In some embodiments, the progression free survival is at least about 45 months. In some embodiments, the progression free survival is at least about 48 months.

In some embodiments, the present invention provides a method of administering niraparib, or a pharmaceutically acceptable salt thereof, to a patient having brain cancer (primary or metastatic) comprising administering niraparib, or a pharmaceutically acceptable salt thereof, according to a regimen determined to achieve a hazard ratio for disease progression or death. In some embodiments, the hazard ratio is improved in patients receiving niraparib, or a pharmaceutically acceptable salt thereof, for example as compared with patients not receiving niraparib, or a pharmaceutically acceptable salt thereof. In some embodiments, the hazard ratio is improved in patients receiving niraparib, or a pharmaceutically acceptable salt thereof, than in patients receiving alternative cancer therapy, for example such as therapy with a different PARP inhibitor. In some embodiments, the invention relates to a method for switching a patient on treatment for cancer with olaparib, or a pharmaceutically acceptable salt thereof, to a treatment comprising niraparib, or a pharmaceutically acceptable salt thereof. In some embodiments, the invention relates to a method for switching a patient on treatment for brain cancer with olaparib, or a pharmaceutically acceptable salt thereof, to a treatment comprising niraparib, or a pharmaceutically acceptable salt thereof. In some embodiments, the invention relates to a method of treating brain cancer (primary or metastatic) comprising administering niraparib, or a pharmaceutically acceptable salt thereof, to a patient who has been previously treated with at least one PARP inhibitor other than niraparib, or a pharmaceutically acceptable salt thereof. In some embodiments, the at least one PARP inhibitor other than niraparib, or a pharmaceutically acceptable salt thereof, includes, but is not limited to, olaparib, pamiparib, rucaparib, and talazoparib, and pharmaceutically acceptable salts thereof. In some embodiments, the PARP inhibitor other than niraparib, or a pharmaceutically acceptable salt thereof, is olaparib, or a pharmaceutically acceptable salt thereof. In some embodiments, the PARP inhibitor other than niraparib, or a pharmaceutically acceptable salt thereof, is pamiparib, or a pharmaceutically acceptable salt thereof. For example, niraparib has demonstrated superior brain penetration when compared to another PARP inhibitor (e.g., olaparib), as evidenced in Examples 3 and 4.

In some embodiments, the brain cancer is primary brain cancer.

In some embodiments, the brain cancer is glioma.

In some embodiments, the brain cancer is unmethylated MGMT glioma. In some embodiments, the brain cancer is unmethylated MGMT glioblastoma.

In some embodiments, the brain cancer is unmethylated glioma. In some embodiments, the brain cancer is unmethylated glioblastoma.

### ADDITIONAL/SUPPORTIVE EMBODIMENTS

1. A method of treating primary or metastatic brain cancer in a human subject in need thereof, the method comprising administering to the human subject an effective dose of niraparib, or a pharmaceutically acceptable salt thereof.
2. The method of embodiment 1, wherein the human subject is also treated with radiotherapy, particularly stereotactic radiation therapy.
3. The method of embodiment 1 or 2, wherein the primary brain cancer or metastatic brain cancer is newly diagnosed.
4. The method of embodiment 2 or 3, wherein the niraparib and radiation therapy begins after resection of the primary brain cancer tumor.
5. The method of any one of embodiments 2-4, wherein the radiation therapy is about 60 Gy (unit gray).
6. The method of any one of embodiments 2-4, wherein the radiation therapy is about 10 Gy (unit gray).
7. The method of any one of embodiments 2-6, wherein the niraparib and radiation therapy is administered for about 6-7 weeks.
8. The method of any one of embodiments 2-7, wherein the human subject further receives maintenance treatment of niraparib without radiation therapy after the treatment with niraparib and radiation therapy.
9. The method of any one of embodiments 1-8, wherein the niraparib, or pharmaceutically acceptable salt thereof, is administered in a dose that is equivalent to 200 mg or 300 mg of niraparib free base.
10. The method of any one of embodiments 1-9, wherein the niraparib, or pharmaceutically acceptable salt thereof, is dosed as niraparib tosylate monohydrate.
11. The method of any one of embodiments 1-10, wherein the niraparib, or pharmaceutically acceptable salt thereof, is administered in the form of a tablet.
12. The method according to any one of embodiments 1-11, wherein the dose of niraparib, or a pharmaceutically acceptable salt thereof, is administered daily.
13. The method according to any one of embodiments 1-12, wherein niraparib, or pharmaceutically acceptable salt thereof, is administered in a daily dose that is equivalent to about 300 mg of niraparib free base.
14. The method according to any one of embodiments 1-12, wherein niraparib, or pharmaceutically acceptable salt thereof, is administered in a daily dose that is equivalent to about 200 mg of niraparib free base.
15. The method of any one of embodiments 1-14, wherein the primary brain cancer is selected from the group consisting of anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumour, pinealoma, diffuse intrinsic pontine glioma, IDH1/2(+) ATRX mutant glioma, malignant glioma and primitive neuroectodermal tumor of the brain.
16. The method of any one of embodiments 1-15, wherein the primary brain cancer is a WHO grade IV tumor.
17. The method of embodiment 16, wherein the primary brain cancer is glioblastoma.
18. The method of embodiment 17, wherein the human subject demonstrates unbound concentrations of niraparib >5-fold of the biochemical IC50 value of niraparib within the gadolinium-nonenhancing region of the tumor of the brain cancer.
19. The method of embodiment 18, wherein the unbound concentrations of niraparib within the gadolinium-nonenhancing region of the tumor of the brain cancer are measured after 4 days of pre-surgical niraparib (300 mg QD) treatment prior to planned resection at 3-5 or 8-12 hours following the last dose.
20. The method of embodiment 18, wherein the unbound concentrations of niraparib within the gadolinium-nonenhancing region of the tumor of the brain cancer are measured after 4 days of pre-surgical niraparib (200 mg QD) treatment prior to planned resection at 3-5 or 8-12 hours following the last dose.
21. The method of any one of embodiments 18-20, wherein 5-fold of the biochemical IC50 value of niraparib is 19 nM.
22. The method of any one of embodiments 1-21, wherein the brain/plasma ratio of niraparib is about 0.5.
23. The method of any one of embodiments 1-22, wherein the niraparib, or pharmaceutically acceptable salt thereof, is administered in combination with one or more additional active agents known to be useful in the treatment of cancer.
24. The method of embodiment 23, wherein the one or more additional active agents is temozolomide, bevacizumab, or a combination thereof.
25. The method according to any one of embodiments 1-24, wherein the human subject or cancer has a complete or partial response to platinum-based chemotherapy.
26. The method according to any one of embodiments 1-25, wherein the cancer is platinum insensitive.
27. The method according to any one of embodiments 1-25, wherein the cancer is platinum sensitive.
28. The method according to any one of embodiments 1-27, wherein the cancer is homologous recombination deficient (HRD) negative.
29. The method of embodiment 1, wherein the primary or metastatic brain cancer is recurrent.
30. The method of embodiment 29, wherein the niraparib administration begins after resection of the brain cancer tumor.
31. The method of embodiment 29 or 30, wherein the niraparib, or pharmaceutically acceptable salt thereof, is administered as maintenance therapy.
32. The method of any one of embodiments 29-31, wherein the niraparib, or pharmaceutically acceptable salt thereof, is administered in a dose that is equivalent to 200 mg or 300 mg of niraparib free base.
33. The method of any one of embodiments 29-32, wherein the niraparib, or pharmaceutically acceptable salt thereof, is dosed as niraparib tosylate monohydrate.
34. The method of any one of embodiments 29-33, wherein the niraparib, or pharmaceutically acceptable salt thereof, is administered in the form of a tablet.
35. The method according to any one of embodiments 29-34, wherein the dose of niraparib, or a pharmaceutically acceptable salt thereof, is administered daily.
36. The method of any one of embodiments 29-35, wherein the primary recurrent brain cancer is selected from the group consisting of anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumour, pinealoma, diffuse intrinsic pontine glioma, IDH1/2(+) ATRX mutant glioma, malignant glioma and primitive neuroectodermal tumor of the brain.
37. The method of any one of embodiments 29-36, wherein the primary recurrent brain cancer is a WHO grade II-IV tumor.
38. The method of any one of embodiments 29-37, wherein the primary recurrent brain cancer is IDH1/2(+) ATRX mutant glioma.
39. The method of embodiment 38, wherein the human subject demonstrates a chromosomal fusion with a cutoff Ct value of 35 in a C-circle assay.
40. The method of embodiment 38, wherein the chromosomal fusion in the C-circle assay is measured after 4 days of pre-surgical niraparib (300 mg QD) treatment prior to planned resection at 3-5 or 8-12 hours following the last dose.
41. The method of any one of embodiments 29-40, wherein the brain/plasma ratio of niraparib is about 0.5.
42. The method of any one of embodiments 29-41, wherein the niraparib, or pharmaceutically acceptable salt thereof, is administered in combination with one or more additional active agents known to be useful in the treatment of cancer.
43. The method of embodiment 42, wherein the one or more additional active agents is temozolomide, bevacizumab, or a combination thereof.
44. The method according to any one of embodiments 29-43, wherein the human subject or cancer has a complete or partial response to platinum-based chemotherapy.
45. The method according to any one of embodiments 29-44, wherein the cancer is platinum insensitive.
46. The method according to any one of embodiments 29-44, wherein the cancer is platinum sensitive.
47. The method according to any one of embodiments 29-46, wherein the cancer is homologous recombination deficient (HRD) negative.
48. The method of any one of embodiments 1-47, wherein the metastatic brain cancer has spread from an original site in the lung, breast, colon, kidney and melanoma.
49. The method of embodiment 48, wherein the metastatic brain cancer is asymptomatic or active progressing brain metastases.
50. The method of embodiment 48 or 49, wherein the metastatic brain cancer is caused by a lung cancer selected from a solid tumor, squamous cell carcinoma of the lung, small cell lung cancer (SCLC), non-small cell lung cancer (NSCLC) and lung adenocarcinoma.
51. The method of embodiment 48 or 49, wherein the metastatic brain cancer is caused by a breast cancer selected from a solid tumor, ductal carcinoma in situ (DCIS; intraductal carcinoma), invasive breast cancer (ILC or IDC; invasive lobular carcinoma or invasive ductal carcinoma), triple negative breast cancer (TNBC), and inflammatory breast cancer.
52. The method of embodiment 48 or 49, wherein the metastatic brain cancer is caused by a kidney cancer selected from a solid tumor, kidney clear cell cancer, kidney papillary cancer, kidney chromophobe cancer, kidney renal cell carcinoma, urothelial carcinoma, kidney sarcoma, Wilms tumor, and kidney lymphoma.
53. The method of embodiment 48 or 49, wherein the metastatic brain cancer is caused by a colon cancer selected from colorectal cancer, squamous cell carcinoma, gastrointestinal neuroendocrine tumors, a solid tumor and adenocarcinoma.
54. The method of embodiment 48 or 49, wherein the metastatic brain cancer is caused by a melanoma selected from superficial spreading melanoma, nodular melanoma, lentigo maligna melanoma, acral lentiginous melanoma, choroidal melanoma, conjunctival melanoma, iris melanoma, and mucosal melanoma.
55. The method of any one of embodiments 48-54, wherein the niraparib administration begins after resection of the brain cancer tumor.
56. The method of embodiment 55, wherein the human subject is also treated with radiotherapy, particularly stereotactic radiation therapy.
57. The method of embodiment 56, wherein the niraparib and radiation therapy begins after resection of the metastatic brain cancer tumor.
58. The method of any one of embodiments 48-57, wherein the niraparib, or pharmaceutically acceptable salt thereof, is administered as maintenance therapy.
59. The method of any one of embodiments 48-58, wherein the niraparib, or pharmaceutically acceptable salt thereof, is administered in a dose that is equivalent to 200 mg or 300 mg of niraparib free base.
60. The method of any one of embodiments 48-59, wherein the niraparib, or pharmaceutically acceptable salt thereof, is dosed as niraparib tosylate monohydrate.
61. The method of any one of embodiments 48-60, wherein the niraparib, or pharmaceutically acceptable salt thereof, is administered in the form of a tablet.
62. The method according to any one of embodiments 48-61, wherein the dose of niraparib, or a pharmaceutically acceptable salt thereof, is administered daily.
63. The method of any one of embodiments 48-62, wherein the metastatic brain cancer comprises asymptomatic and active progressing brain metastases.
64. The method of any one of embodiments 48-63, wherein the brain/plasma ratio of niraparib is about 0.5.
65. The method of any one of embodiments 48-64, wherein the niraparib, or pharmaceutically acceptable salt thereof, is administered in combination with one or more additional active agents known to be useful in the treatment of cancer.
66. The method of embodiment 65, wherein the one or more additional active agents is temozolomide, bevacizumab, or a combination thereof.
67. The method according to any one of embodiments 48-66, wherein the human subject or cancer has a complete or partial response to platinum-based chemotherapy.
68. The method according to any one of embodiments 48-66, wherein the cancer is platinum insensitive.
69. The method according to any one of embodiments 48-66, wherein the cancer is platinum sensitive.
70. The method according to any one of embodiments 48-69, wherein the cancer is homologous recombination deficient (HRD) negative.
71. The method according to any one of embodiments 1-70, comprising administering niraparib, or a pharmaceutically acceptable salt thereof, to a patient who has been previously treated with at least one PARP inhibitor other than niraparib, or a pharmaceutically acceptable salt thereof.
72. The method according to embodiment 71, wherein the at least one PARP inhibitor other than niraparib, or a pharmaceutically acceptable salt thereof, is selected from the group consisting of olaparib, pamiparib, rucaparib, and talazoparib, and pharmaceutically acceptable salts thereof.
73. The method according to embodiment 71, wherein the at least one PARP inhibitor other than niraparib, or a pharmaceutically acceptable salt thereof, is olaparib, or a pharmaceutically acceptable salt thereof.

### EXAMPLES

The following examples are provided to illustrate, but not limit the claimed invention.

### Example 1. a Phase 0 ‘Trigger’ Trial of Niraparib in Newly-Diagnosed Glioblastoma and Recurrent IDH1/2(+) ATRX Mutant Glioma

This is an open-label, multi-center Phase 0 study with an expansion phase that will enroll up to 24 participants with newly-diagnosed glioblastoma and up to 18 recurrent glioma participants with IDH mutation and ATRX loss. As described below, the trial will be composed of a Phase 0 component (subdivided into Arm A and B) and a therapeutic expansion phase. Patients with tumors demonstrating a positive PK Response (in Arm A) or a positive PD Response (in Arm B) of the Phase 0 component of the study will graduate to a therapeutic expansion phase that combines therapeutic dosing of niraparib plus standard-of-care fractionated radiotherapy (in Arm A) or niraparib monotherapy (in Arm B) until progression of disease.

Arm A will accrue patients at the Ivy Brain Tumor Center only whereas Arm B will be open at the Ivy Brain Tumor Center plus UCSF Medical Center.

### Phase 0

### Arm A:

Participants undergoing resection for a presumed newly-diagnosed glioblastoma (WHO grade 4) will be treated with niraparib for 4 days prior to surgical resection. Up to 24 participants will receive presurgical niraparib in anticipation of arriving at 12 participants in the therapeutic expansion phase (see below). The final presurgical dose will be administered 3-5 hours or 8-10 hours before tumor resection. Assignment to time-cohorts will be random. Participants who do not demonstrate histologically-proven diagnosis of glioblastoma following the craniotomy will be replaced. In each of the two time-cohorts, tumor PK and PD analyses will be performed at the Ivy Brain Tumor Center.

For PK analysis, blood, CSF, and brain tumor tissue samples (gadolinium enhancing and non-enhancing) will be collected intraoperatively. Additional blood samples will be obtained on Day 1 pre-dose and on Day 4 (the day of the surgery) at pre-dosing (trough level), 0.5, 1, 2, 4, 6, 9-, 12-, 24-, and 48-hours post-dose.

For PD analysis, intraoperatively-collected gadolinium-enhancing tumor tissue will be sectioned into 4 similarly-sized tissue portions. Two tissue specimens will be exposed to 10 Gy of IR using a RAD2000 device and two specimens will be sham irradiated (non-irradiated). One pair of sham control and 10 Gy radiated tissue will be fixed in formalin solution for FFPE slides. The other pair will be snap-frozen for PAR ELISA assay. Additional gadolinium-enhancing tumor tissue will be cryopreserved for genomic analyses.

TABLE 1
Time Cohorts

| Time Cohort | N= | Drug | Surgical Time |
|---|---|---|---|
| Cohort 1 | 3 | 300 mg QD of | Day 4: 3-5 hrs post |
| | | Niraparib for 4 days | last dose |
| Cohort 2 | 3 | 300 mg QD of Niraparib | Day 4: 8-10 hrs post |
| | | for 4 days | last dose |

The Optimal Time-Interval (OTI) is the surgical time interval (STI) from final presurgical dose to tumor resection to detect maximal unbound drug concentration in nonenhancing tumor. The OTI will be determined after 3 participants in each cohort are enrolled. Once the OTI is determined, the remaining 18 participants in Arm A will be enrolled in the OTI, either 3-5 hours or 8-10 hours after the last dose. Arm B will enroll into the OTI determined in Arm A.

### Pharmacokinetics (PK) Methods

Unbound niraparib concentration in gadolinium-non-enhancing glioblastoma tissue is the primary endpoint for this Phase 0 trial. In the Ivy Center's CLIA-certified PK Core, total and unbound concentration of niraparib in plasma, CSF, and gadolinium-enhancing and gadolinium-non-enhancing regions of tumor will be assessed for each patient. Tumor-to-plasma ratios will be calculated. All assays and analyses will be developed, validated, and performed at Ivy Brain Tumor Center.

For graduation to the therapeutic expansion phase of the study, a positive PK Response must be demonstrated and is defined as unbound concentrations of niraparib >5-fold of the biochemical IC50 value within the gadolinium-nonenhancing region of the tumor.

### Pharmacodynamics (PD) Methods

A ‘functional’ PD assay examining PAR activity in the setting of ex vivo irradiation will serve as a secondary endpoint in this protocol. In the Ivy Center's CLIA-certified PD Core, FFPE embedded tumor tissue from both ex vivo irradiated and non-irradiated to assess changes in PAR, cleaved caspase 3, γH2AX and Ki67 using immunohistochemistry (IHC) assays. Alternatively, ELISA assays to quantify PAR levels will be performed using frozen lysates from the second pair of control and irradiated samples.

### Arm B:

Enrollment in Arm B will begin after 6 participants (3 in each time cohort) have been enrolled in Arm A and Optimal Time Interval (OTI) has been determined. Participants undergoing resection of a recurrent WHO Grade II, III, or IV glioma with IDH1 or IDH2 mutation and ATRX loss will be treated with niraparib for 4 days prior to a planned surgical resection. Up to 18 participants will receive presurgical niraparib in anticipation of arriving at 12 participants within the therapeutic expansion phase. The final dose will be administered at the OTI before the surgical specimens are collected. Following resection, participants who do not have histologically-proven tumor recurrence (e.g., pseudoprogression) will be replaced. For patients in each of the two time-cohorts, tumor PK and PD analyses will be performed.

For PK analysis, blood, CSF, and brain tumor tissue samples (gadolinium enhancing and non-enhancing) will be collected intraoperatively. Additional blood samples will be obtained on Day 1 pre-dose and on Day 4 (the day of the surgery) at pre-dosing (trough level), 0.5, 1, 2, 4, 6, 9-, 12-, 24-, and 48-hours post-dose.

For PD analysis, intraoperatively-collected tumor tissue will be snap-frozen for assessing the chromosomal fusion as a primary endpoint. Additional exploratory assays including c-circle assay and immunohistochemistry for MIB-1, Cleaved Caspase-3, H2AX will be performed. If archival or pre-surgical biopsy is obtained, they will be used for baseline comparison. Additional intraoperatively-collected tumor tissue will be cryopreserved for genomic analyses.

TABLE 2
Time Cohort

| | N= | Drug | Surgical Time |
|---|---|---|---|
| Arm B | 18 | 300 mg QD of | Day 4: OTI post |
| | | Niraparib for 4 days | last dose |

### Pharmacokinetics (PK) Methods

Unbound niraparib concentration in gadolinium-nonenhancing glioma tissue is the secondary endpoint in Arm B of this protocol. In the Ivy Center's CLIA-certified PK Core, total and unbound concentration of niraparib in plasma, CSF, and gadolinium-enhancing and gadolinium-non-enhancing regions of tumor will be assessed for each patient. Tumor-to-plasma ratios will be calculated. All assays and analyses will be developed, validated, and performed at Ivy Brain Tumor Center.

### Pharmacodynamics (PD) Methods

PCR based quantification of chromosomal fusion will serve as a primary endpoint in Arm B of this protocol. In the Ivy Center's CLIA-certified PD Core, frozen lysates from intraoperative tumor tissue will be utilized to extract gDNA. 25 ng of gDNA will be used for qPCR-based detection of chromosomal fusion. A C-circle assay will be performed as an exploratory endpoint. The quantification of MIB-1, γH2AX and Cleaved Caspase-3 from immunohistochemistry assays will be performed.

For enrollment to the expansion component of the study, a positive PD Response is defined as the presence of the chromosomal fusion with the cutoff Ct value of 35.

### Therapeutic Expansion Phase:

### Arm A:

Patients with tumors that have an unmethylated MGMT promoter and demonstrate a positive PK response (see above) in the Phase 0 component of Arm A will enter the therapeutic expansion phase of this study. These participants will receive niraparib in combination with radiation (60 Gy over 6-7 weeks, as per standard of care).
For patients weighing <77 kg (<170 lbs) OR with a platelet count <150,000/mcL, the recommended dosage is 200 mg taken orally once daily.
For patients weighing ≥77 kg (≥170 lbs) AND a platelet count >150,000/mcL, the recommended dosage is 300 mg taken orally once daily.

Following radiotherapy, study participants may receive niraparib maintenance treatment which will begin after a 4-week rest period (+7 days) to allow for recovery from RT plus niraparib treatment. Participants will continue receiving maintenance treatment until progression of disease, unacceptable toxicity or death, withdrawal of consent, loss to follow-up, or study termination by sponsor.

### Arm B:

Patients with tumors that demonstrate a positive PD Response (see above) in the Phase 0 component of Arm B will enter the expansion component of this study. Participants will receive niraparib until progression of disease, unacceptable toxicity or death, withdrawal of consent, loss to follow-up, or study termination by sponsor.
For patients weighing <77 kg (<170 lbs) OR with a platelet count <150,000/mcL, the recommended dosage is 200 mg taken orally once daily.
For patients weighing ≥77 kg (≥170 lbs) AND a platelet count ≥150,000/mcL, the recommended dosage is 300 mg taken orally once daily.

All participants (Arm A and B) will return to the clinic to monitor safety per the schedule of events until treatment is ended and will then be contacted approximately every 3 months by letter or phone for collection of survival data for up to 24 months. The start of follow up for long-term survival begins following completion of the Day 28 safety follow-up visit.

Additional biomarker analysis will be conducted using surgical tissue. If the participant undergoes repeat craniotomy or biopsy for recurrence or progression of his/her brain tumor, Ivy Brain Tumor Center will request samples from the resected tumor to enable longitudinal sample collection and analysis that will help identify possible resistance mechanisms.

### Primary Objectives

1. Phase 0:
Arm A: To evaluate the relative pharmacokinetics (PK) of niraparib in tumor tissue from glioblastoma participants treated with niraparib.
Arm B: To evaluate the pharmacodynamics (PD) impact of niraparib in tumor tissue from WHO Grade 2-4 patients.
2. Expansion: Examine progression-free survival (6 months) in participants with demonstrated PK (Arm A) and PD (Arm B) effects.

### Primary Endpoints

1. Phase 0:
Arm A: For PK analysis, total and unbound niraparib concentration in Gd enhancing and Gd-non-enhancing tumor tissue. Intra-op tumor (enhancing and non-enhancing tissue) to plasma (collected during surgery) partition coefficients of niraparib for total (Kp) and unbound (Kp, uu) drug levels will be evaluated.
Arm B: For PD analysis, presence of chromosomal fusion in niraparib treated glioma tissue with IDH and ATRX loss.
2. Expansion: Examine progression-free survival rate measured from time of surgery to date of recurrence.

### Exploratory Objectives

1. Phase 0: To evaluate the additional pharmacodynamics (PD) biomarkers of niraparib.
2. Phase 0: To evaluate the relative pharmacokinetics (PK) of niraparib in CSF.

### Exploratory Endpoints

1. Phase 0: For Arm A: quantification of γH2AX, ClCas3 and Ki67 positive cells will be summarized in ex-vivo irradiated samples.

For Arm B, quantification of c-circles, γH2AX, ClCas3 and Ki67 positive cells will be summarized.
2. Phase 0: Niraparib level in intraoperative CSF (3-5 hr and 8-10 hr) will be determined.

### Study Population:

Arm A: Patients undergoing planned resection for a suspected newly diagnosed glioblastoma.
Arm B: Patients undergoing planned resection for a recurrent WHO Grade 2-4 glioma with IDH mutation and ATRX loss.

Each participant must meet all the following inclusion criteria and none of the exclusion criteria to be eligible for study entry.

### Inclusion Criteria:

1. Arm A participants undergoing resection for a suspected newly diagnosed glioblastoma. For Arm B, participants undergoing resection who have had a prior resection of histologically diagnosed WHO grade II-IV glioma with IDH1 or IDH2 mutation and ATRX loss.
2. Arm A participants must have measurable disease preoperatively, defined as at least 1 contrast-enhancing lesion, with 2 perpendicular measurements of at least 1 cm.
3. Ability to understand and the willingness to sign a written informed consent document (personally or by the legally authorized representative, if applicable).
4. Participant has voluntarily agreed to participate by giving written informed consent (personally or via legally authorized representative(s), and assent if applicable). Written informed consent for the protocol must be obtained prior to any screening procedures. If consent cannot be expressed in writing, it must be formally documented and witnessed, ideally via an independent trusted witness.
5. Willingness and ability to comply with scheduled visits, treatment plans, laboratory tests and other procedures.

$$6. Age \geq 18 at time of consent$$

$$7. Have a performance status ( PS ) of \leq 2 on the Eastern Cooperative Oncology Group ( ECOG ) scale ( Oken et al . 1982 )$$

8. Ability to swallow oral medications.
9. Confirmed negative serum pregnancy test (β-hCG) before starting study treatment or participant who is no longer of childbearing potential due to surgical, chemical, or natural menopause. If the serum pregnancy test is completed >7 days from Day 1, a urine pregnancy test will be done to confirm a negative result prior to Day 1 dose administration.
10. For females of reproductive potential: use of highly effective contraception for at least 1 month prior to treatment and agreement to use such a method during study participation and for an additional 3 months after the end of treatment administration.
11. For males of reproductive potential: use of condoms or other methods to ensure effective contraception with partner and for an additional 3 months after the end of treatment administration. Avoid sperm donation for duration of the study and for an additional 3 months after the end of treatment administration.
12. Agreement to adhere to Lifestyle Considerations throughout study duration.
13. Participants who received chemotherapy must have recovered (Common Terminology Criteria for Adverse Events [CTCAE] Grade ≤1) from the acute effects of chemotherapy except for residual alopecia or Grade 2 peripheral neuropathy prior to Day 1. A washout period of at least 21 days (or more per investigator discretion) is required between last chemotherapy and Day 1.
14. Females of child-bearing potential must agree not to breastfeed starting at screening, throughout the study period and for 6 months after final study drug administration.
15. Participant has normal blood pressure or adequately treated and controlled hypertension (Defined as systolic BP≤140 mmHg and diastolic BP≤90 mmHg).
16. Participant has adequate bone marrow and organ function as defined by the following laboratory values (as assessed by the local laboratory for eligibility):

### Adequate Bone Marrow Function:

$$Absolute Neutrophil Count \geq 1 , 500 / mcL$$

$$Platelets ( at time of surgery ) \geq 100 , 000 / mcL$$

$$Hemoglobin \geq 9. g / dL$$

Participants may receive erythrocyte transfusions to achieve this hemoglobin level at the discretion of the investigator. Initial treatment must not begin earlier than the day after the erythrocyte transfusion.

### Adequate Hepatic Function:

Total Bilirubin ≤1.5×ULN. Participants with Gilbert's syndrome with a total bilirubin ≤2.0 times ULN and direct bilirubin within normal limits are permitted.

$$AST ( SGOT ) \leq 2.5 \times institutional ULN$$

$$ALT ( SGPT ) \leq 2.5 \times institutional ULN$$

Adequate Renal Function: Estimated glomerular filtration rate (eGFR) ≥30 mL/min/1.73 m2 by Chronic Disease Epidemiology Collaboration (CKD-EPI) equation

$$INR \leq 1.5 \times ULN$$

### Exclusion Criteria:

1. Current use of coumarin-derived anticoagulant for treatment, prophylaxis or otherwise, that cannot be discontinued prior to surgery. Therapy with heparin, low molecular weight heparin (LMWH) or fondaparinux is allowed.
2. Pregnancy or lactation.
3. Known allergic reactions to components of the niraparib tablet, including FD&C Yellow No. 5.
4. Active infection or fever >38.5° C. requiring systemic antibiotic, antifungal or antiviral therapy within 4 weeks of Day 1.
5. Known to have active (acute or chronic) or uncontrolled severe infection, liver disease such as cirrhosis, decompensated liver disease, and active and chronic hepatitis as determined by the investigator.
6. Known active systemic bacterial infection (requiring intravenous [IV] antibiotics at time of initiating study treatment), fungal infection, or detectable viral infection (such as known human immunodeficiency virus positivity or with known active hepatitis B or C [for example, hepatitis B surface antigen positive]. Screening is not required for enrollment.
7. Any of the following cardiovascular criteria:
   Current evidence of cardiac ischemia
   Current symptomatic pulmonary embolism
   Acute myocardial infarction ≤6 months prior to Day 1
   Heart failure of New York Heart Association Classification III or IV ≤6 months prior to Day 1

$$Grade \geq 2 ventricular arrhythmia \leq 6 months prior to Day 1$$

$$Cerebral vascular accident ( CVA ) or transient ischemic attack ( TIA ) \leq 6 months prior to Day 1$$

8. Participant has myelodysplastic syndrome/acute myeloid leukemia or with features suggestive of MDS/AML.
9. Participant has serious and/or uncontrolled preexisting medical condition(s) that, in the judgment of the investigator, would preclude participation in this study (for example, interstitial lung disease, severe dyspnea at rest or requiring oxygen therapy, severe renal impairment], history of major surgical resection involving the stomach or small bowel, or preexisting Crohn's disease or ulcerative colitis or a preexisting chronic condition resulting in baseline Grade 2 or higher diarrhea).
10. Prior therapy with PARP inhibitors at a therapeutic dose.
11. Treatment with another investigational drug or other intervention within 5 half-lives of the investigational product.
Phase: Phase 0 with therapeutic expansion phase

### Description of Sites/Facilities Enrolling Participants:

Arm A: One U.S. Site will enroll participants for this study.
Arm B: Two U.S. sites.

### Description of Study Intervention:

### Phase 0:

For Arm A, niraparib administered orally QD for 4 days prior to surgical resection. The last dose will be the AM dose on Day 4, at 3-5 hours or 8-10 hours, prior to the planned resection. For Arm B, niraparib administered orally QD for 4 days prior to surgical resection. The last dose will be the AM dose on Day 4, at 3-5 hours or 8-10 hours, prior to planned resection.

### Expansion:

Arm A: Participants (from Phase 0) with positive PK and unmethylated MGMT promoter: niraparib administered orally QD continuously (7 days/week) in combination with 6-7 weeks of RT 5 days/week. Day 1 of each cycle will occur as the first day of radiation therapy.
Arm B: niraparib orally QD will be taken by the participants (from Phase 0) with positive PD as long as the drug is tolerated, and the investigator believes the participant may be obtaining benefit. Treatment will be taken by the participant until confirmed progression or end of treatment.
Both Arms will receive niraparib as follows:

For patients weighing <77 kg (<170 lbs) OR with a platelet count <150,000/mcL, the recommended dosage is 200 mg taken orally once daily.

For patients weighing ≥77 kg (≥170 lbs) AND a platelet count ≥150,000/mcL, the recommended dosage is 300 mg taken orally once daily.

### Maintenance Phase (Arm A):

Niraparib (administered continuously Days 1-28).
   For patients weighing <77 kg (<170 lbs) OR with a platelet count <150,000/mcL, the recommended dosage is 200 mg taken orally once daily.
   For patients weighing ≥77 kg (≥170 lbs) AND a platelet count ≥150,000/mcL, the recommended dosage is 300 mg taken orally once daily.

### Study Duration:

The estimated total study length of ˜38 months includes 2 months for protocol preparation (inclusive of IRB/IND approval), 12 months for trial accrual of 24 newly-diagnosed unmethylated glioblastoma patients (Arm A) and 18 recurrent glioma (Arm B), and 24 months of follow-up for participants in the expansion phase.

The Ivy Brain Tumor Center operates on approximately 600 gliomas per year. Approximately 150 are newly-diagnosed WHO Grade IV gliomas. Of the 400 recurrent glioma operations annually, >90% are recurrent WHO Grade II-IV gliomas.

UCSF Medical Center operates on approximately 400 gliomas per year. Approximately 100 are newly-diagnosed WHO Grade IV gliomas. Of the 300 recurrent glioma operations annually, >90% are recurrent WHO Grade II-IV gliomas.

### Participant Duration:

Phase 0: up to approximately 2 months (screening window of 28 days through Day 28 follow-up visit).
Expansion (Arm A): up to approximately 6 weeks for treatment.
Expansion (Arm B): niraparib will be taken by the participant as long as the drug is tolerated, and the investigator believes the participant may be obtaining benefit. Treatment will be taken by the participant until confirmed progression or end of treatment.
Maintenance (Arm A): niraparib will be taken by the participant as long as the drug is tolerated, and the investigator believes the participant may be obtaining benefit. Treatment will be taken by the participant until confirmed progression or end of treatment.

All participants in Arm A will be followed for survival up to 24 months.

All participants in Arm B will be followed for survival up to 48 months.

### Statistical Considerations:

The primary objective is an exploratory study to evaluate the PK of niraparib in Arm A and PD of niraparib in Arm B and to examine progression-free survival (PFS6) in both Arms, and no formal statistical hypothesis tests will be performed. The sample size was justified based on feasibility and previous studies from our group. In Arms A, up to 12 participants will be assigned for each time cohort and in Arm B, up to nine participants will be assigned in each time cohort. The planned sample size for phase 0 will be up to 24 newly-diagnosed glioblastoma patients for Arm A and 18 recurrent glioma patients with IDH and ATRX mutations in Arm B. The proportions of positive PK response for each cohort will be reported for phase 0 primary endpoint for PK analysis. To compare PD biomarkers, ex vivo processed tumor tissue will be examined with and without radiation treatment. Once the percentage of individual biomarker positive cells are quantified, a paired t test will be used to compare log-transformed percent positive cells between treated and untreated tissue at a 2-sided 5% level. The chi-square test and normal approximation method and corresponding 95% confidence intervals based on proportion difference on PFS6 will be calculated for expansion phase participants. Logrank tests will be applied to compare overall survivals between two treatments with Kaplan-Meier curve analysis. Baseline characteristics, including demographics and laboratory measurements, will be summarized using descriptive statistics. Mean, Median, Minimum and Maximum values will be reported for continuous values such as age and laboratory values, and N and % will be reported for categorical values such as sex and performance status.

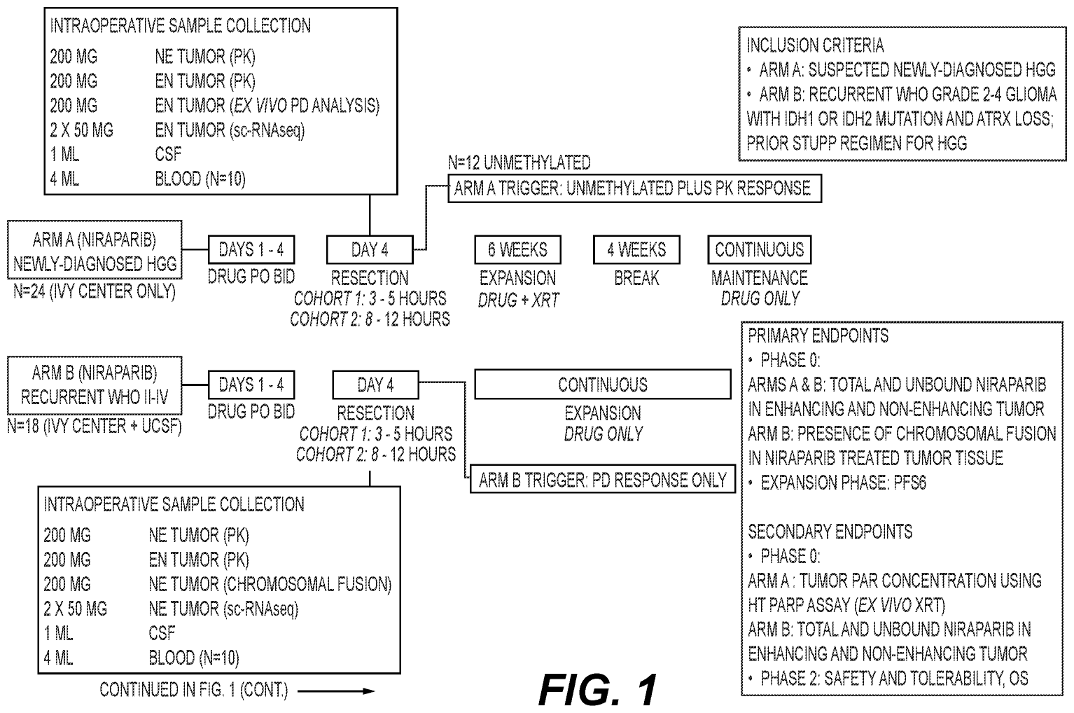

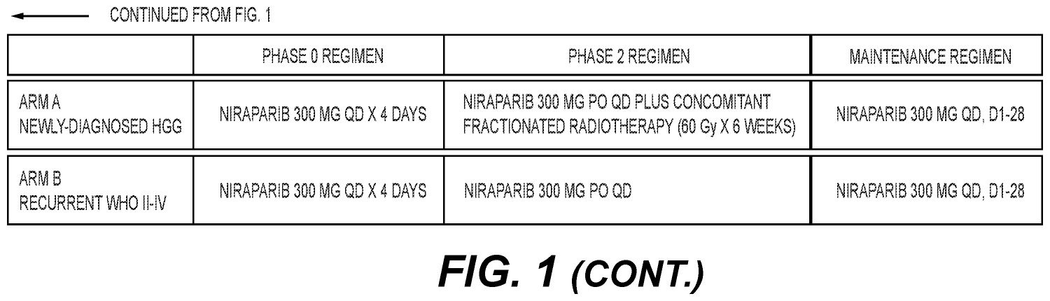

The protocol for the Phase 0 Arm A and Arm B studies is summarized in FIG. 1.

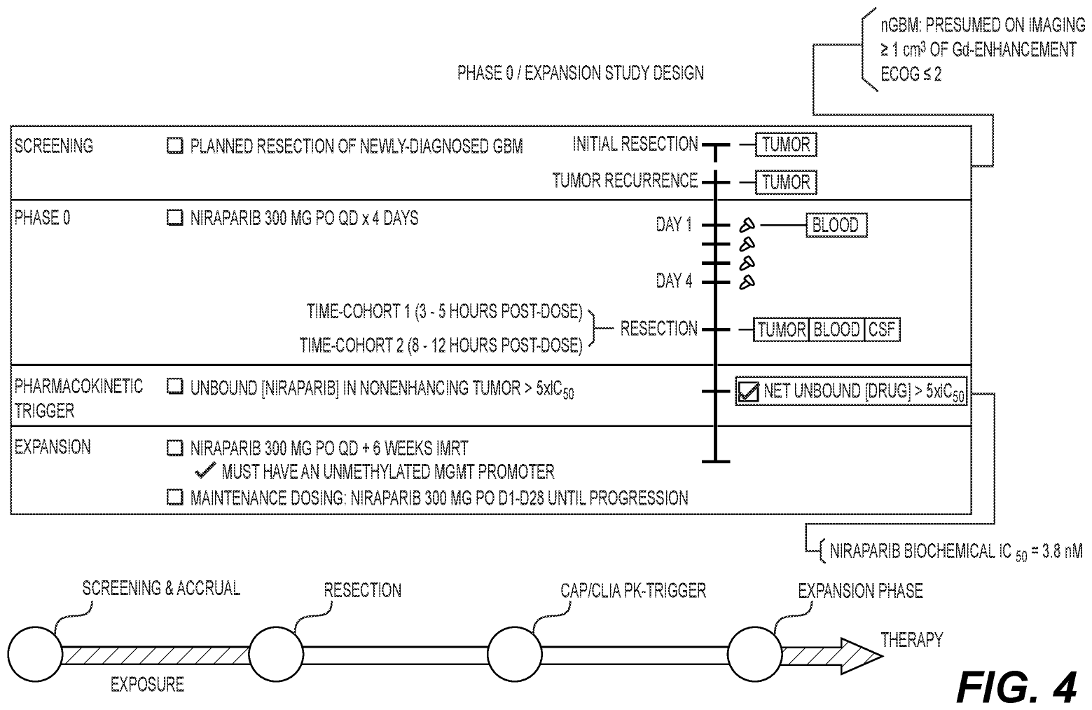

The protocol for Arm A is further summarized in FIG. 4.

### Interim Results Arm A

METHODS: Newly-diagnosed GBM patients received 4 days of pre-surgical niraparib (300 mg QD) prior to planned resection at 3-5 hours or 8-12 hours following the last dose. Tumor tissue (enhancing and nonenhancing regions), cerebrospinal fluid (CSF), and plasma were collected. Total and unbound niraparib concentrations were measured using validated LC-MS/MS methods. A PK ‘trigger’ determined eligibility for the therapeutic expansion phase and was defined as unbound [niraparib]>5-fold biochemical IC50 (i.e., 19 nM) in non-enhancing tumor. Evidence of PARP inhibition was assessed by 10 Gy ex vivo irradiation of surgical tissue and quantification of PARylation compared to non-irradiated control tissue. Patients with 06-Methylguanine-DNA Methyltransferase (MGMT)-unmethylated tumors exceeding the PK threshold were eligible for expansion phase dosing of niraparib plus fractionated radiotherapy followed by a maintenance phase of niraparib monotherapy.

RESULTS: Twelve patients were enrolled into the Phase 0 study with two patients continuing to the expansion phase. During Phase 0, one patient experienced a Grade 3 treatment-related adverse event (ALT and AST elevation) and, during the expansion phase, two patients demonstrated treatment-related thrombocytopenia. The mean unbound concentrations of niraparib in Gd-nonenhancing tumor region were 340.9 nM and 331.9 nM in the 3-5 hr cohort (n=8) and the 8-12 hr (n=3) cohort, respectively. All tumors met the PK criteria for the expansion phase and two demonstrated unmethylated-MGMT. Fold increase in PAR levels after ex vivo radiation compared to untreated samples was 2.44 in nGBM control (n=4) vs 1.71 in study patients (n=10), respectively.

CONCLUSIONS: This first-in-human study of niraparib in newly-diagnosed GBM patients (nGBM) demonstrates brain tumor penetration in excess of any other studied PARP inhibitor and concomitant radiosensitization via PAR suppression.

### Additional Interim Results Arm A

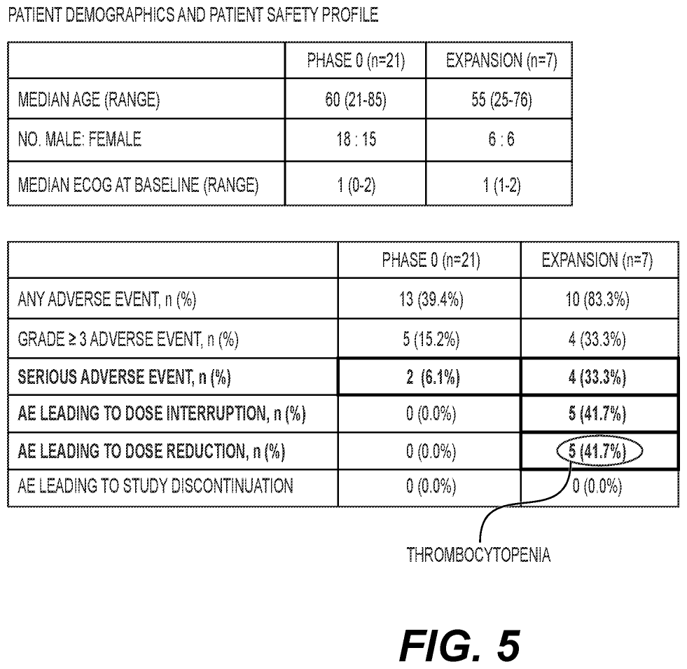

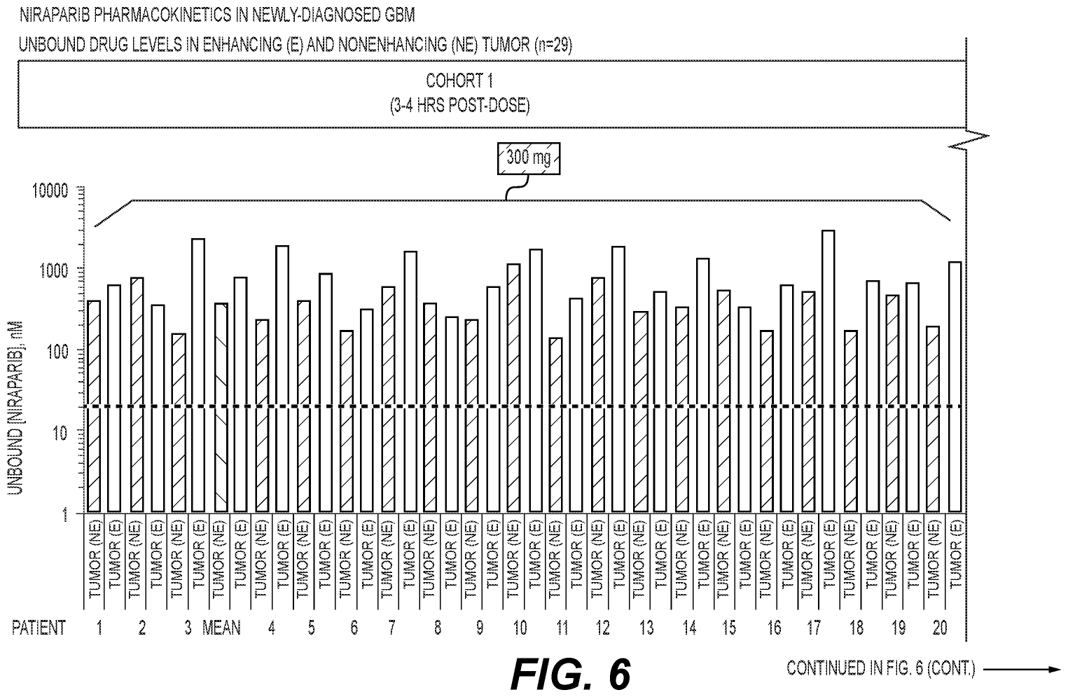

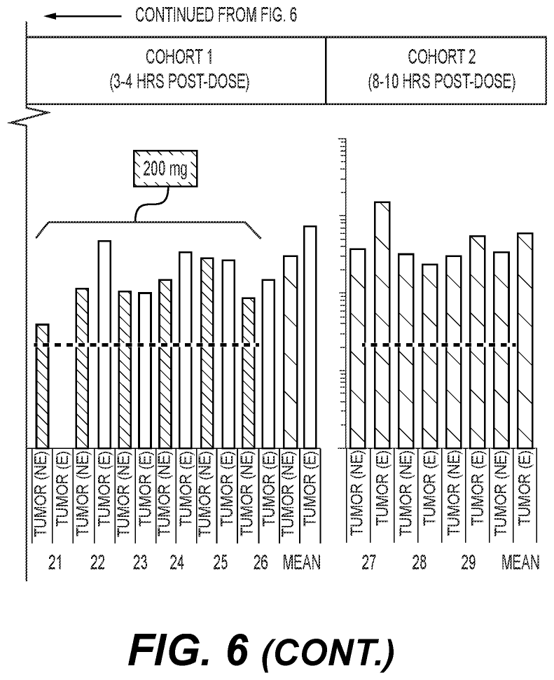

DEMOGRAPHICS AND SAFETY DATA: Thirty-three patients (45.4% female) participated in Arm A of Phase 0, and twelve patients advanced to the expansion phase, and the demographics and safety data for the patients is summarized in FIG. 5. In the Phase 0 component of the trial, the median age (range) of patients in Arm A was 60 (21-85) years, with a median ECOG at baseline (range) of 1 (0-2).

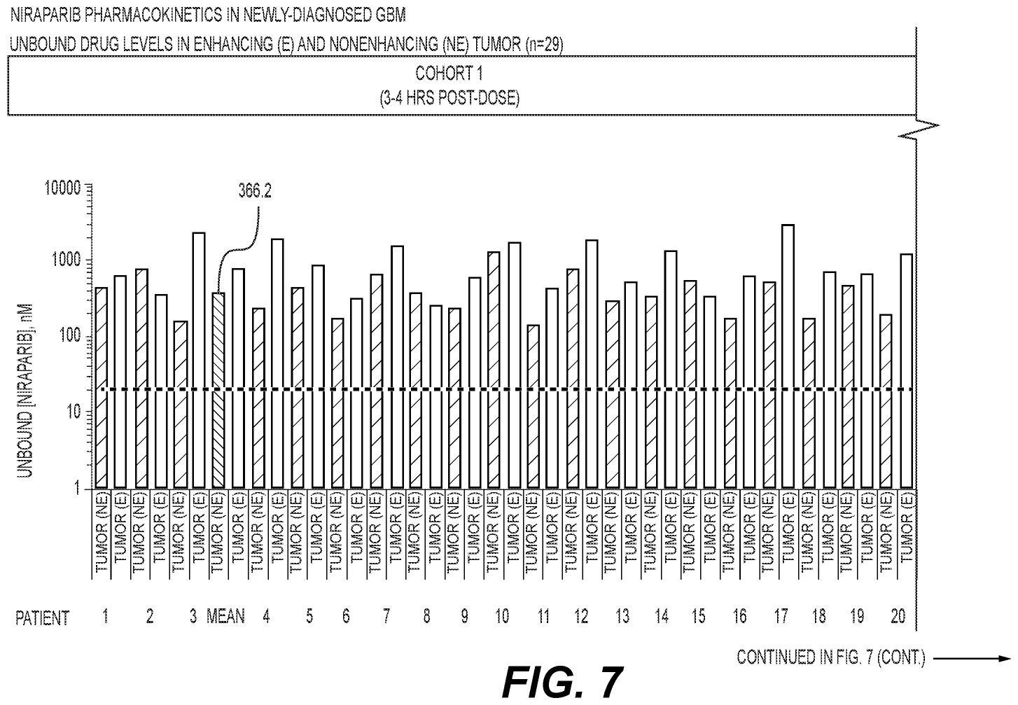

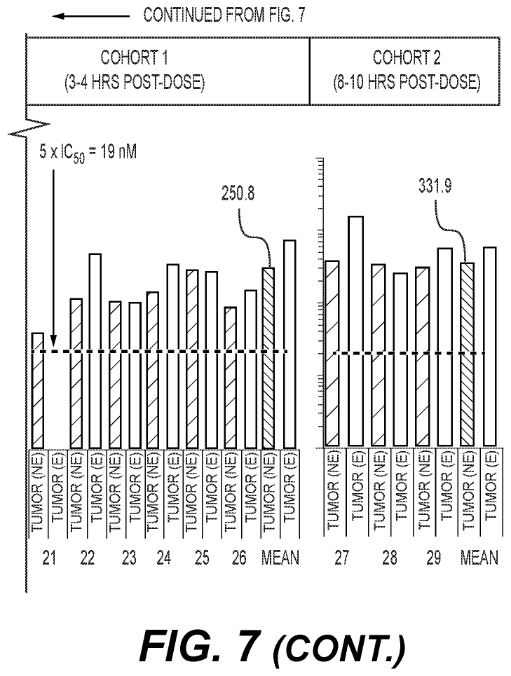

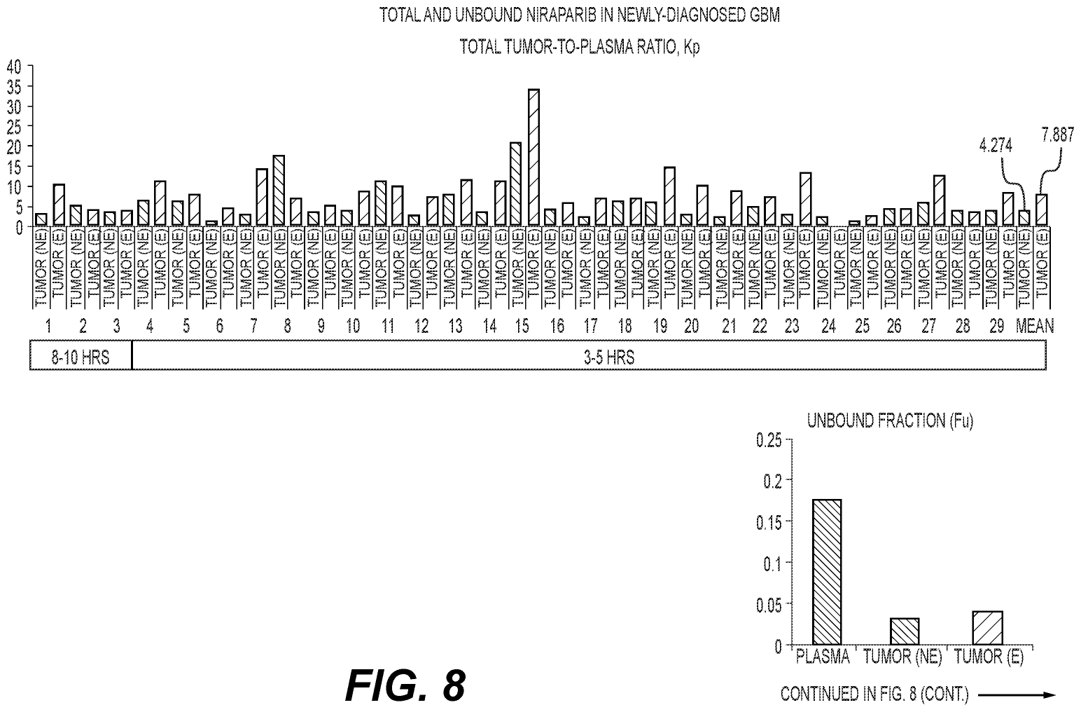

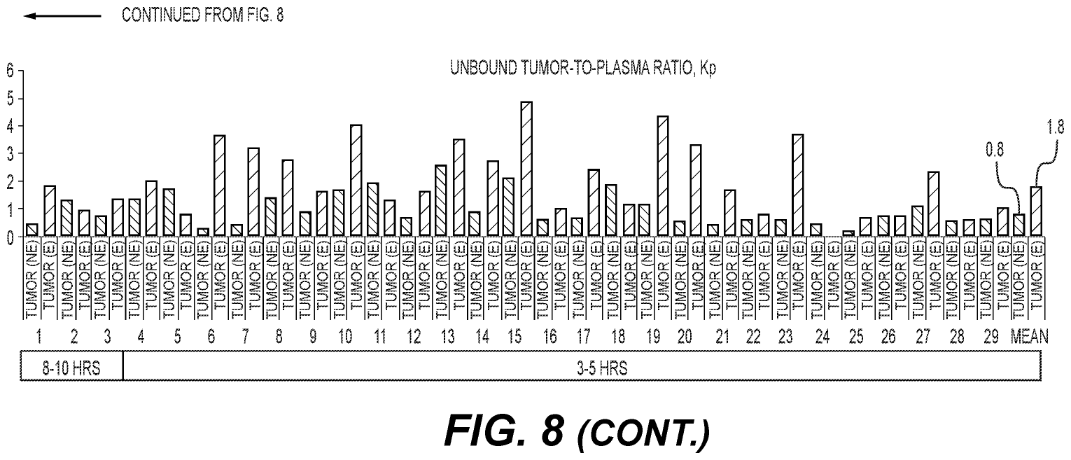

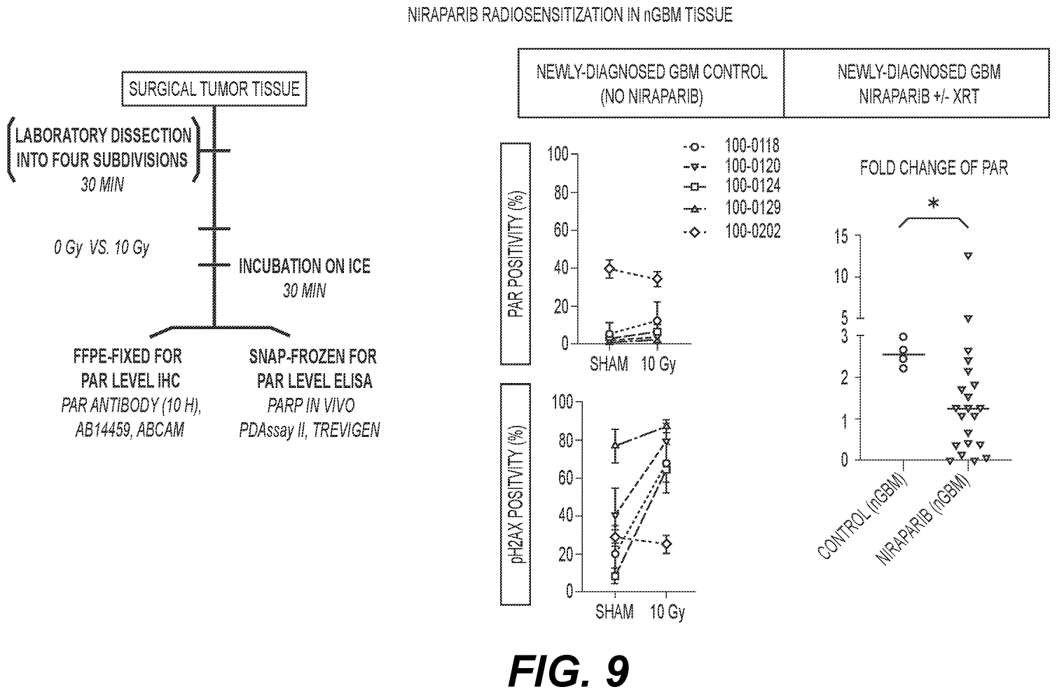

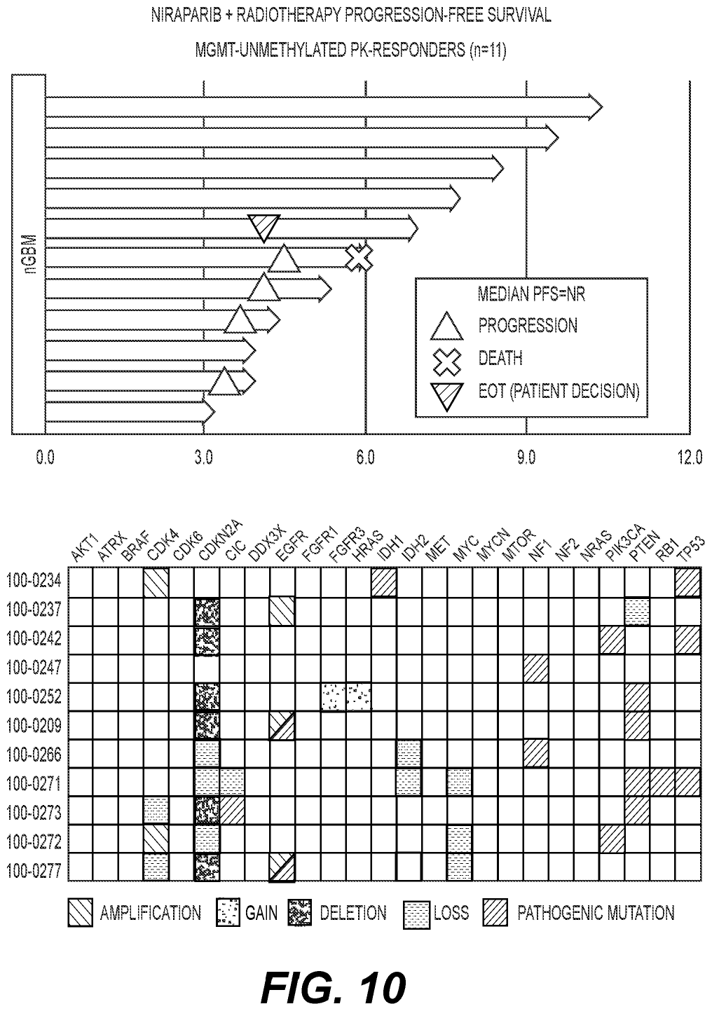

RESULTS: The mean unbound concentrations of niraparib in Gd-nonenhancing tumor region of the 3-5 hr cohort 1 patients were 250.8 nM and 366.2 nM in the 200 mg (n=6) and 300 mg (n=20) dosed patients, respectively, as indicated in FIG. 7. The mean unbound concentration of niraparib in Gd-nonenhancing tumor region in the 8-12 hr cohort 2 patients was 331.9 nM in the 300 mg dosed patients (n=3), as indicated in FIG. 7. These unbound concentrations of niraparib were considerably above the IC₅₀ of niraparib (i.e., 19 nM). The total tumor:plasma (Kp) ratio of niraparib was 4.274 and 7.887 in non-enhancing and enhancing tissue, respectively, as shown in FIG. 8. The unbound tumor:plasma ratio (Kp, uu) was 0.8 and 1.8 in non-enhancing and enhancing tissue, respectively, as shown in FIG. 8. Fold increase in PAR levels after ex vivo radiation compared to untreated samples was over 2.5 in nGBM control vs. below 1.5 in study patients, respectively, as shown in FIG. 9. Three or four days of niraparib exposure suppressed induction of post-irradiation poly(ADP-ribose) (PAR) levels in ex-vivo newly-diagnosed and recurrent GBM tissue. Clinical outcomes for the MGMT-unmethylated PK responder expansion patients are summarized in FIG. 10. MGMT-unmethylated PK-responders were dosed niraparib in addition to radiotherapy. Of the 6 patients with ≥6 months follow-up, four are in remission and two had a recurrence of tumor.

CONCLUSIONS: Niraparib was generally well-tolerated in newly-diagnosed and recurrent glioblastoma patients. Niraparib reached pharmacologically-relevant concentrations in non-enhancing newly-diagnosed and recurrent glioblastoma tissue at 200 mg and 300 mg dosing. Three or four days of niraparib exposure suppressed induction of post-irradiation PAR levels in ex vivo newly-diagnosed and recurrent glioblastoma tissue.

### Further Interim Results Arm A

MATERIALS/METHODS: Patients with presumed newly-diagnosed GBM were enrolled in a phase 0 study receiving 4 days of niraparib (300 or 200 mg QD) prior to planned resection 3-5 or 8-12 hours following the last dose. Tumor tissue (enhancing and non-enhancing regions), cerebrospinal fluid (CSF), and plasma were collected. Total and unbound niraparib concentrations were measured using validated LC-MS/MS methods. PARP inhibition was assessed by quantification of PAR induction after 10 Gy ex vivo irradiation in surgical tissue compared to non-irradiated control tissue. A PK ‘trigger’ determined eligibility for the therapeutic phase 2 expansion portion of the study. This was defined as unbound [niraparib]>5-fold biochemical IC50 (i.e., 19 nM) in non-enhancing tumor. Patients with MGMT unmethylated tumors exceeding this PK threshold were eligible for expansion phase dosing of niraparib with concurrent RT followed by a maintenance phase of niraparib. Patients with MGMT methylated tumors were not eligible for the expansion phase and proceeded with temozolomide (TMZ) plus RT followed by maintenance TMZ. RT dose was 60 Gy in 30 fractions using volumetric-modulated arc therapy (VMAT).

RESULTS: All 29 patients enrolled in the phase 0 portion of the study met the PK threshold. The unbound concentrations of niraparib were considerably above the IC₅₀ of niraparib. In non-enhancing regions, the mean unbound concentration of niraparib was 258.2 nM. The suppression of PAR levels after ex vivo radiation was observed in 79% of the patients (17/22). Sixteen patients had unmethylated tumors, and of those, 11 patients enrolled in phase 2. Five of the 6 initial patients enrolled in phase 2 experienced thrombocytopenia related to niraparib, and 3/5 cases were deemed serious and life-threatening. Consequently, starting dose in both phases was lowered to 200 mg, and no serious AEs were observed thereafter. At a median follow-up of 8.1 months [range: 6.0-12.9 months], PFS6 was 64% with 4 patients remaining on treatment and 5 patients ongoing survival follow-up.

CONCLUSIONS: Niraparib achieves pharmacologically-relevant concentrations in non-enhancing, newly-diagnosed GBM tissue. When delivered with concurrent RT, niraparib was well-tolerated, with low rates of grade 3+ toxicity. Initial clinical efficacy data are encouraging.

### Example 2. Investigation of the Brain Penetration of GSK3985771C (Niraparib) in the P-Gp (P-Glycoprotein/Mdr1-a) and BCRP (Breast Cancer Resistance Protein/ABCG2) Rat Knock-Out Model

### Methods:

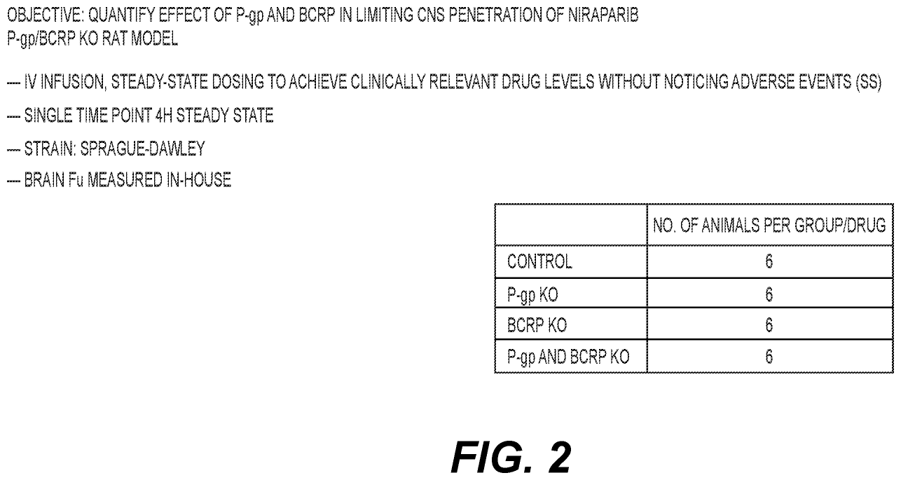

At least three days prior to the start of the study, six male rats per group (total 24 rats) received surgically implanted femoral vein and carotid artery catheters for infusion of the test molecule and blood sampling, respectively. Doses were filtered prior to administration and the actual dose administered to the animals was quantified. All PK parameters were calculated based on the actual dose administered to each animal. Animals received an intravenous bolus loading dose followed by a constant intravenous infusion to maintain steady state of drug level. At the end of the infusion, animals were euthanized and brain tissues were collected. Tissue homogenates were prepared in PBS (1:4 w/v). LC-MS/MS method was used to quantitate drug concentrations. Tissue homogenate concentrations were corrected for test compound contained in residual plasma in the tissue. The tissue-to-plasma ratio was calculated by dividing corrected tissue concentration by steady-state plasma concentration. In vitro protein binding of niraparib in rat plasma and brain homogenate was assessed by rapid equilibrium dialysis method. Data are reported as fraction unbound (Fu). The protocol is summarized in FIG. 2.

### Conclusion:

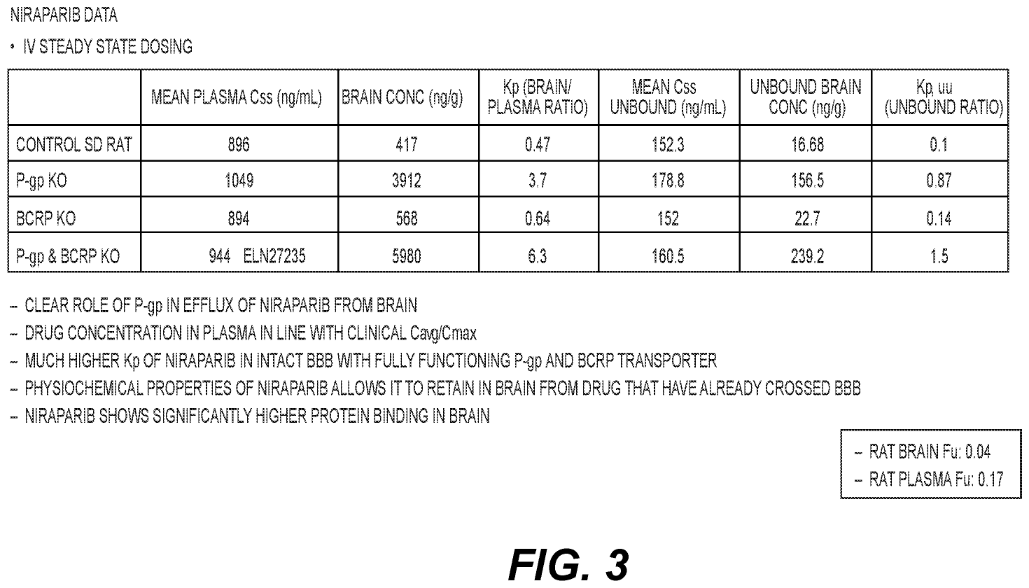

Niraparib plasma concentrations in all four groups tested were almost similar. Niraparib brain penetration in control rats was almost half of that in plasma. Brain tissue concentrations further increased 9-fold and 14-fold in Mdr1-a (P-gp) knockout and Pgp/BCRP double knockout, respectively, while BCRP knockout rats had <2-fold increase. Brain:plasma (Kp) ratio was 0.47, 3.7, 0.64 and 6.3 in control, P-gp knock-out, BCRP knock-out and P-gp/BCRP double knock-out rat model, respectively. Unbound fraction of niraparib was 0.17 and 0.04 in rat plasma and brain, respectively. Unbound brain:plasma ratio (Kp, uu) was 0.1, 0.87, 0.15 and 1.5 control, P-gp knock-out, BCRP knock-out and P-gp/BCRP double knock-out rat model respectively. Even though there is a role of P-gp in limiting the niraparib penetration into the brain; the brain exposures of niraparib in control rat with fully functioning P-gp and BCRP appears to show favorable profile for niraparib as a potential therapeutic agent to treat brain tumors. These results are summarized in FIG. 3.

### Example 3. Differentiation of Niraparib and Olaparib Brain Penetration in a Mouse Brain Metastatic Tumor Model

There remains an unmet need to provide effective treatment therapy for patients with primary and metastatic brain tumors; mainly due to lack of drug penetration across the blood brain barrier (BBB). Synthetic lethality remains an attractive mechanism in treating brain tumors post radiotherapy. In this study, brain penetration and distribution of niraparib and olaparib in a mouse brain tumor model were evaluated.

Female mice (CrTac:NCr-Foxnlnu; 6 w/o) received 2.5E5 luciferase transfected human breast cancer line (MDA 231-BRM2-831) via intracardiac injection. Mice were imaged twice/week using bioluminescence imaging (BLI) to monitor tumor growth. On day 35, mice with brain metastases (BM) were treated via oral gavage once daily for 5 days with either niraparib (35 mg/kg, n=4 BM, n=3 control), olaparib (50 mg/kg, n=3 BM, n=3 control), or vehicle (n=3 control). Terminal blood samples and brains were collected 2 hr post final dose. Serial tissue sections were collected for MALDI-IMS, H&E and IHC staining from 5 distinct horizontal planes in the brain. Tissue collected between each imaging plane was homogenized for LC-MS bioanalysis.

In vivo BLI imaging was used to identify mice with brain metastases (BM) and the presence of tumors throughout the brains were confirmed ex vivo using IHC. Quantitative ex vivo imaging analysis by MALDI IMS of coronal brain sections collected from mice administered niraparib showed consistent concentrations distributed throughout the brain parenchyma with locally higher concentrations detected from tumor regions. Table 3 summarizes the LC-MS bioanalysis concentrations in plasma and bulk brain homogenates and brain section concentrations detected by MALDI IMS. The estimated mean unbound brain-to-plasma partition coefficient (Kp,uu,brain) was 3.0× and 5.6× higher for niraparib compared to olaparib in control and BM mice, respectively.

These results demonstrated that niraparib has a higher brain penetration compared to olaparib in both control mice and mice with BM.

TABLE 3
Summary of LC-MS Bioanalysis and
MALDI IMS quantification results

| | | LC-MS Bioanalysis | | MALDI | |
|---|---|---|---|---|---|
| | | Terminal | | IMS | |
| | | Plasma | Bulk Brain | Brain | |
| | | Avg | Homogenate | Section | |
| | Animal | (ng/mL) | Avg | Median | K_{p, uu Brain} |
| Niraparib | Control | 3100 (620) | 658 (111) | 532 (89) | 0.18 |
| | BM | 2535 (507) | 543 (91) | 473 (80) | 0.18 |
| | Vehicle | ND | ND | | |
| Olaparib | Control | 322 (99) | 12 (2) | ND | 0.02 |
| | BM | 254 (78) | 11 (2) | ND | 0.03 |
| | Vehicle | ND | ND | | |

Values are total concentrations with adjusted free concentration in parentheses
K_{p, uu brain} calculated using bulk brain homogenate and terminal plasma free concentrations

### Example 4. Differentiation of Niraparib and Olaparib Brain Penetration in Healthy Rhesus Macaque Monkeys

There remains an unmet need to provide effective treatment therapy for patients with primary and metastatic brain tumors; mainly due to lack of drug penetration across the blood brain barrier (BBB). Synthetic lethality remains an attractive mechanism in treating brain tumors post radiotherapy. This study investigates the brain penetration of niraparib and olaparib in healthy monkeys to generate evidence of niraparib's ability to cross the blood brain barrier (BBB).

Four healthy male Rhesus macaque monkeys were dosed daily via oral gavage for five days with either niraparib (n=2; 6 mg/kg) or olaparib (n=2; 10 mg/kg). Pre-dose blood was collected daily and terminal blood, cerebrospinal fluid (CSF), and brain tissue was collected at necropsy. Coronal brain sections were analyzed by matrix-assisted laser desorption/ionization (MALDI) imaging mass spectrometry (IMS) to quantitatively assess the tissue distribution of the dosed compounds. Blood, CSF, and bulk homogenates of brain tissue were analyzed by LC-MS bioanalysis.

Referring to Table 4, greater brain penetration was observed for niraparib when compared to olaparib in healthy Rhesus macaque monkeys following five days oral administration. The unbound brain-to-plasma partition coefficient (Kp,uu,brain) was 15× higher for niraparib compared to olaparib. Quantitative ex vivo imaging analysis by MALDI IMS of coronal brain sections collected from monkeys administered niraparib showed consistent concentrations distributed throughout the brain parenchyma. Olaparib was not detected by MALDI IMS in any of the coronal brain sections collected from the monkeys administered olaparib. Similar plasma and CSF concentrations were observed between the monkey's administered niraparib vs those administered olaparib, highlighting the unique ability of niraparib to cross the intact blood brain barrier of monkeys and distribute throughout the brain.

Niraparib showed markedly higher brain penetration than olaparib in healthy Rhesus macaque monkeys demonstrating enhanced ability to cross intact BBB.

TABLE 4
Summary of LC-MS Bioanalysis and MALDI IMS quantification results

| | | | | | | | | MALDI | | | |
|---|---|---|---|---|---|---|---|---|---|---|---|
| | | LC-MS Bioanalysis | | | | | | IMS | | | |
| | | Pre-Dose | | | | | | Brain | | | |
| | | Plasma | Terminal | | Terminal | Bulk Brain | | Section | | | |
| | | Days 1-5 | Plasma | | CSF | Homogenate | | Average | | | Mean |
| | Animal | (ng/mL) | (ng/mL) | | (ng/mL) | (ng/g) | | (ng/g) | | K_{p, uu Brain/Plasma} | K_{p, uu Brain/Plasma} |
| Niraparib | Monkey | BQL, 28 | 84 | (31) | 9 | 378 | (14) | 283 | (10) | 0.45 | 0.30 |
| | 1 | (10), 49 | | | | | | | | | |
| | | (18), 59 | | | | | | | | | |
| | | (22), 72 | | | | | | | | | |
| | | (26) | | | | | | | | | |
| | Monkey | BQL, 58 | 436 | (160) | 25 | 797 | (29) | 684 | (25) | 0.16 | |

| | | | | | | | | | | |
|---|---|---|---|---|---|---|---|---|---|---|
| | 2 | (21), 87 | | | | | | | | |
| | | (32), 74 | | | | | | | | |
| | | (27), 66 | | | | | | | | |
| | | (24) | | | | | | | | |
| Olaparib | Monkey | BQL, 27 | 322 | (99) | 13 | 12 | (2) | BLQ | 0.02 | 0.02 |
| | 1 | (8), 26 (8), | | | | | | | | |
| | | 33 (10), 33 | | | | | | | | |
| | | (10) | | | | | | | | |
| | Monkey | BQL, 51 | 254 | (78) | 6 | 11 | (2) | BLQ | 0.03 | |

| | |
|---|---|
| 2 | (16), 17 |
| | (5), 53 |
| | (16), 45 |
| | (14) |

Values are total concentrations with adjusted free concentration in parentheses
K_{p, uu brain} calculated using bulk brain homogenate and terminal plasma free concentrations

### EQUIVALENTS

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

1. A method of treating primary or metastatic brain cancer in a human subject in need thereof, the method comprising administering to the human subject an effective dose of niraparib, or a pharmaceutically acceptable salt thereof.

2. The method of claim 1, wherein the human subject is also treated with radiotherapy, particularly stereotactic radiation therapy.

3. The method of claim 1, wherein the primary brain cancer or metastatic brain cancer is newly diagnosed.

4. The method of claim 2, wherein the administration of niraparib, or a pharmaceutically acceptable salt thereof, and radiation therapy begins after resection of the primary brain cancer tumor.

5. The method of claim 2, wherein the radiation therapy is about 60 Gy (unit gray).

6. The method of claim 2, wherein the radiation therapy is about 10 Gy (unit gray).

7. The method of claim 2, wherein the niraparib, or a pharmaceutically acceptable salt thereof, and radiation therapy is administered for about 6-7 weeks.

8. The method of claim 2, wherein the human subject further receives maintenance treatment of niraparib, or a pharmaceutically acceptable salt thereof, without radiation therapy after the treatment with niraparib and radiation therapy.

9. The method of claim 1, wherein the niraparib, or a pharmaceutically acceptable salt thereof, is administered in a dose that is equivalent to 200 mg or 300 mg of niraparib free base.

10. The method of claim 1, wherein the niraparib, or pharmaceutically acceptable salt thereof, is dosed as niraparib tosylate monohydrate.

11. The method of claim 1, wherein the niraparib, or pharmaceutically acceptable salt thereof, is administered in the form of a tablet.

12. The method of claim 1, wherein the dose of niraparib, or a pharmaceutically acceptable salt thereof, is administered daily.

13. The method of claim 1, wherein niraparib, or pharmaceutically acceptable salt thereof, is administered in a daily dose that is equivalent to about 300 mg of niraparib free base.

14. The method of claim 1, wherein niraparib, or pharmaceutically acceptable salt thereof, is administered in a daily dose that is equivalent to about 200 mg of niraparib free base.

15. The method of claim 1, wherein the primary brain cancer is selected from the group consisting of anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumor, pinealoma, diffuse intrinsic pontine glioma, IDH1/2(+) ATRX mutant glioma, malignant glioma, and primitive neuroectodermal tumor of the brain.

16. The method of claim 1, wherein the primary brain cancer is a WHO grade IV tumor.

17. The method of claim 15, wherein the primary brain cancer is glioblastoma.

18. The method of claim 1, wherein the human subject demonstrates unbound concentrations of niraparib >5-fold of the biochemical IC50 value of niraparib in tumor tissue of the brain cancer.

19. The method of claim 18, wherein the human subject demonstrates unbound concentrations of niraparib >5-fold of the biochemical IC50 value of niraparib in non-enhancing or enhancing tumor tissue of the brain cancer.

20. The method of claim 19, wherein the unbound concentrations of niraparib in the non-enhancing or enhancing tumor tissue of the brain cancer are measured after pre-surgical niraparib treatment.

21. The method of claim 1, wherein the human subject demonstrates unbound concentrations of niraparib >5-fold of the biochemical IC50 value of niraparib within the gadolinium-nonenhancing region of the tumor of the brain cancer.

22. The method of claim 18, wherein the unbound concentrations of niraparib in tumor tissue are measured post-resection.

23. The method of claim 21, wherein the unbound concentrations of niraparib within the gadolinium-nonenhancing region of the tumor of the brain cancer are measured after 4 days of pre-surgical niraparib (300 mg QD) treatment prior to planned resection at 3-5 or 8-12 hours following the last dose.

24. The method of claim 21, wherein the unbound concentrations of niraparib within the gadolinium-nonenhancing region of the tumor of the brain cancer are measured after 4 days of pre-surgical niraparib (200 mg QD) treatment prior to planned resection at 3-5 or 8-12 hours following the last dose.

25. The method of claim 18, wherein the unbound concentrations of niraparib are measured in brain tumor tissue samples collected intraoperatively.

26. The method of claim 18, wherein 5-fold of the biochemical IC50 value of niraparib is 19 nM.

27. The method of claim 1, wherein the brain/plasma ratio of niraparib is about 0.5.

28. The method of claim 1, wherein the niraparib, or a pharmaceutically acceptable salt thereof, is administered in combination with one or more additional active agents known to be useful in the treatment of cancer.

29. The method of claim 28, wherein the one or more additional active agents is temozolomide, bevacizumab, or a combination thereof.

30. The method of claim 1, wherein the human subject or cancer has a complete or partial response to platinum-based chemotherapy.

31. The method of claim 30, wherein the cancer is platinum insensitive.

32. The method of claim 30, wherein the cancer is platinum sensitive.

33. The method of claim 1, wherein the cancer is homologous recombination deficient (HRD) negative.

34. The method of claim 1, wherein the metastatic brain cancer has spread from an original site in the lung, breast, colon, kidney or melanoma.

35. The method of claim 1, comprising administering niraparib, or a pharmaceutically acceptable salt thereof, to a patient who has been previously treated with at least one PARP inhibitor other than niraparib, or a pharmaceutically acceptable salt thereof.

36. The method of claim 35, wherein the at least one PARP inhibitor other than niraparib, or a pharmaceutically acceptable salt thereof, is selected from the group consisting of olaparib, pamiparib, rucaparib, and talazoparib, and pharmaceutically acceptable salts thereof.

37. The method of claim 36, wherein the at least one PARP inhibitor other than niraparib, or a pharmaceutically acceptable salt thereof, is olaparib, or a pharmaceutically acceptable salt thereof.
